(19) United States
(12) Patent Application Publication
(10) Pub. No.: US 2013/0058930 A1
(43) Pub. Date: Mar. 7, 2013

(54) SUBCUTANEOUS NEEDLE ASSISTED JET INJECTION ADMINISTRATION OF METHOTREXATE

(71) Applicants: Kaushik J. DAVE, Edison, NJ (US); Peter L. SADOWSKI, Woodbury, MN (US); Paul K. WOTTON, Newtown, PA (US); John W. HAYES, Chaska, MN (US)

(72) Inventors: Kaushik J. DAVE, Edison, NJ (US); Peter L. SADOWSKI, Woodbury, MN (US); Paul K. WOTTON, Newtown, PA (US); John W. HAYES, Chaska, MN (US)

(73) Assignee: ANTARES PHARMA, INC., Minneapolis, MN (US)

(21) Appl. No.: 13/564,693

(22) Filed: Aug. 1, 2012

Related U.S. Application Data
(60) Provisional application No. 61/514,112, filed on Aug. 2, 2011.

(51) Int. Cl. A61K 31/519; A61P 37/06; A61K 39/395

(52) U.S. Cl. 424/134.1; 514/249; 424/133.1

(57) ABSTRACT

The present application is directed, at least in part, to a method of treating an autoimmune disorder in a subject in need of treatment. In one exemplary embodiment, the method comprises introducing into the subcutaneous tissue of the subject, from a needle assisted jet injection device, a composition comprising methotrexate in a dose ranging from about 5 mg to about 50 mg, wherein the pharmacokinetic profile of said methotrexate, obtained following administration of the methotrexate by the needle assisted jet injection device, is substantially the same as the pharmacokinetic profile of the same dose of methotrexate when administered by an intramuscular injection or a subcutaneous injection. The present invention provides benefits and improvements, including an improved clinical utility, improved therapeutic efficacy, over conventional methods of administering methotrexate.

### RELATED APPLICATIONS

This application is a U.S. non-provisional of and claims priority benefit from U.S. Provisional Application No. 61/514,112, filed Aug. 2, 2011, which is incorporated herein by reference in its entirety.

### FIELD OF THE INVENTION

The present invention generally relates to methods of treating autoimmune and inflammatory disorders, said methods including the use of a needle assisted jet injection device for delivery of a composition comprising methotrexate and/or a pharmaceutically acceptable salt thereof, by targeting said composition to the subcutaneous compartment of a subject's skin.

### BACKGROUND OF THE INVENTION

Methotrexate (formerly Amethopterin) is an antimetabolite used in the treatment of certain neoplastic diseases, severe psoriasis, and adult rheumatoid arthritis. The International Union of Pure and Applied Chemistry (“IUPAC”) nomenclature for methotrexate is N-[4-[[(2,4-diamino-6-pteridinyl)methyl]methylamino]benzoyl]-L-glutamic acid. Methotrexate has the following structural formula.

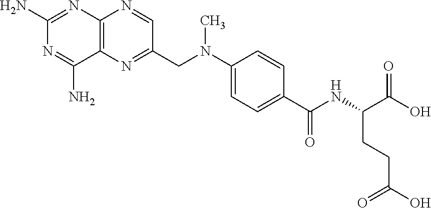 [SMILES: CN(Cc1cnc2nc(N)nc(N)c2n1)c1ccc(C(=O)N[C@@H](CCC(=O)O)C(=O)O)cc1]

In the United States, the Food and Drug Administration approved indications for use of Methotrexate include treatment of neoplastic diseases, including gestational choriocarcinoma, chorioadenoma destruens and hydatidiform mole; psoriasis, in particular, in the symptomatic control of severe, recalcitrant, disabling psoriasis; and management of selected adults with severe, active rheumatoid arthritis (ACR criteria), or children with active polyarticular-course juvenile rheumatoid arthritis.

Injectable solutions of methotrexate of various strengths, with or without preservatives, for single use only or for multiple-use, are available from a number of drug manufacturers, including Hopsira, Inc., Sandoz, Inc. and Medac GmbH. Hospira-Methotrexate Injection, USP, isotonic liquid, contains preservative, with each 25 mg/ml, 2 ml vial containing methotrexate sodium equivalent to 50 mg methotrexate. Hospira-Methotrexate Injection, USP, isotonic liquid, preservative free, 10 mg/ml, 2 ml single-dose vial contains methotrexate sodium equivalent to 20 mg methotrexate. Hospira-Methotrexate Injection, USP, isotonic liquid, preservative free, single use 25 mg/ml, 20 ml, 40 ml and 100 ml vial contains methotrexate sodium equivalent to 500 mg, 1 g and 2.5 g methotrexate respectively.

Sandoz-Methotrexate Injection, USP (preservative free) is supplied in a single-dose vial containing 25 mg/ml of methotrexate as the base in the following package strengths: 50 mg in 2 ml multi-dose vial (packaged in 10's), 250 mg in 10 ml (packaged in 10's), and 1 gram in 40 ml single-dose vial (individually packaged).

Medac GmbH's Metoject® 10 mg/ml is a ready to use pre-filled syringe for injection containing methotrexate and is provided with an injection needle. Metoject® 10 mg/ml, has been marketed by Medac GmbH since 2000. It can be injected intravenously, intramuscularly (into a muscle, e.g. thigh) or subcutaneously.

In 2008, the Medical Products Agency (MPA) granted marketing authorization for Metoject® 50 mg/ml solution for injection to Medac Gesellschaft Far and Klinische Spezialpräparate MBH. Like Metoject® 10 mg/ml, Metoject® 50 mg/ml, solution for injection, is ready to use pre-filled syringe containing methotrexate and is provided with an injection needle. MPA, is the Swedish national authority responsible for regulation and surveillance of the development, manufacturing and marketing of drugs and other medicinal products in Sweden and in other European contracting states.

After reviewing and assessing an application submitted by Medac GmbH in support of its request for marking Metoject® 50 mg/ml in Sweden, MPA published its decision and findings in the form of a public assessment report. According to the MPA's public assessment report for Metoject® 50 mg/ml, “[t]he administration of the higher strength (50 mg/mL) of MTX [methotrexate] resulted in similar total exposure in terms of AUC, but somewhat higher Cₘₐₓ (15-20% higher), compared with the marketed lower strength (10 mg/mL) following both i.m. and s.c. administration.”

Accordingly, an urgent need exists for more efficient methods of administering injectable low and high strength solution of methotrexate that achieves pharmacokinetics, including systemic bioavailability, and Cₘₐₓ, that is reliably consistent regardless whether the same dose of methotrexate is administered via a subcutaneous or an intramuscular route, to provide benefits and improvements, including an improved clinical utility, improved therapeutic efficacy to patients, over conventional methods of administering methotrexate to patients.

### BRIEF SUMMARY OF THE INVENTION

In accordance with one aspect of the present invention, there is provided a method of treating an autoimmune or inflammatory disorder in a subject in need of treatment with a pharmaceutical preparation. According to an exemplary embodiment, the method comprises introducing into the subcutaneous tissue of a subject, from a needle assisted jet injection device, a composition comprising methotrexate in a dose ranging from about 5 mg to about 50 mg, wherein the pharmacokinetic profile of said methotrexate delivered by said needle assisted jet injection device is substantially the same as the pharmacokinetic profile of the same dose of said methotrexate when administered to said subject using an intramuscular injection or a subcutaneous injection. In an embodiment, said pharmacokinetic profile comprises bioavailability of said methotrexate. In another embodiment, said pharmacokinetic profile comprises the time of peak concentration (Tₘₐₓ) of the blood (serum or plasma) concentration-time curve of said methotrexate. In yet another embodiment, said pharmacokinetic profile comprises peak height concentration (Cₘₐₓ) of the blood (or serum or plasma) concentration time curve of said methotrexate. In another embodiment, said pharmacokinetic profile comprises the area under the blood (serum or plasma) concentration-time curve (AUC) of said methotrexate.

In accordance with another aspect of the present invention, there is provided a method of treating an autoimmune or inflammatory disorder in a subject in need of treatment with methotrexate. In an exemplary embodiment, said method comprises introducing through a subject's skin and into the subcutaneous tissue of said subject a composition comprising methotrexate, wherein said methotrexate is in a dose ranging from about 5 mg to about 50 mg, wherein said methotrexate has a substantially the same pharmacokinetic profile when introduced into the subcutaneous tissue of the subject using a needle assisted jet injection device, or using an intramuscular injection or a subcutaneous injection. In an embodiment, said pharmacokinetic profile obtained by introducing said methotrexate from said needle assisted jet injection device comprises a Cₘₐₓ for said methotrexate, when assayed in the blood (plasma or serum) of said subject following said introduction by said needle assisted jet injection device, that is substantially the same as the Cₘₐₓ for said methotrexate when administered at the same dose using an intramuscular injection or a subcutaneous injection. In another embodiment, said pharmacokinetic profile comprises an AUC for said methotrexate, when assayed in the blood (plasma or serum) of said subject following said introduction by said needle assisted jet injection device, that is substantially the same as the AUC for said methotrexate when administered at the same, dose using an intramuscular injection or a subcutaneous injection. In yet another embodiment, said pharmacokinetic profile comprises a Tₘₐₓ for said methotrexate, when assayed in the blood (plasma or serum) of said subject following said introduction by said needle assisted jet injection device that is substantially the same as the Tₘₐₓ for said methotrexate when administered at the same dose using an intramuscular injection or a subcutaneous injection. In another embodiment, said pharmacokinetic profile comprises any combination of Tₘₐₓ, Cₘₐₓ and AUC for methotrexate.

In an embodiment, said autoimmune disorder is selected from the group consisting of Juvenile idiopathic arthritis (JIA), Juvenile rheumatoid arthritis (JRA), Psoriatic arthritis (PA), and Rheumatoid arthritis (RA). In another embodiment, said autoimmune disorder is selected from the group consisting of Juvenile rheumatoid arthritis (JRA), and Rheumatoid arthritis (RA). In yet another embodiment, said autoimmune disorder is Juvenile rheumatoid arthritis (JRA). In another embodiment, said autoimmune disorder is Rheumatoid arthritis (RA). In an embodiment, said autoimmune disorder is moderate to severe rheumatoid arthritis with at least one of one condition selected from pain, stiffness, swelling, fatigue and combinations thereof. In an embodiment, said inflammatory disorder is cardiovascular disease associated with inflammation. In an embodiment, said inflammatory disorder is due to atherosclerotic plaque associated with foam cells. In an embodiment said inflammatory disorder is cardiovascular disease associated with rheumatoid arthritis. In an embodiment of the method of the present invention, the pharmacokinetic profile provides a mean Cₘₐₓ of about 213 ng/ml when the dose of methotrexate is about 10 mg. In another embodiment, the pharmacokinetic profile provides an AUC₍₀₋ₜ₎ of about 1141 ng*hr/ml when the dose of methotrexate is about 10 mg. In another embodiment, the pharmacokinetic profile provides an AUC₍₀₋₂₄₎ of about 1150 ng*hr/ml when the dose of methotrexate is about 10 mg. In another embodiment, the pharmacokinetic profile provides an AUC_{(0-inf)} of about 1161 ng*hr/ml when the dose of methotrexate is about 10 mg. In another embodiment, the pharmacokinetic profile provides a Tₘₐₓ of about 1.33 hours when the dose of methotrexate is about 10 mg. In another embodiment, the pharmacokinetic profile provides a half-life of about 3 hours when the dose of methotrexate is about 10 mg. In another embodiment, the pharmacokinetic profile provides a mean Cₘₐₓ of from about 170 ng/ml to about 266 ng/ml when the dose of methotrexate is about 10 mg. In another embodiment, the pharmacokinetic profile provides an AUC₍₀₋ₜ₎ of from about 912 ng*hr/ml to about 1426 ng*hr/ml when the dose of methotrexate is about 10 mg. In another embodiment, the pharmacokinetic profile provides an AUC₍₀₋₂₄₎ of from about 920 ng*hr/ml to about 1437 ng*hr/ml when the dose of methotrexate is about 10 mg. In another embodiment, the pharmacokinetic profile provides an AUC_{(0-inf)} is from about 929 ng*hr/ml to about 1451 ng*hr/ml when the dose of methotrexate is about 10 mg. In another embodiment, the pharmacokinetic profile a Tₘₐₓ of from about 1.06 hours to about 1.66 hours when the dose of methotrexate is about 10 mg. In another embodiment, the pharmacokinetic profile provides a half-life of from about 2.6 hours to about 4.06 hours when the dose of methotrexate is about 10 mg. In another embodiment, the pharmacokinetic profile provides a mean Cₘₐₓ of about 356 ng/ml when the dose of methotrexate is about 15 mg. In another embodiment, the pharmacokinetic profile provides an AUC_{(0-inf)} of about 1945 ng*hr/ml when the dose of methotrexate is about 15 mg. In another embodiment, the pharmacokinetic profile provides an AUC₍₀₋₂₄₎ of about 1948 ng*hr/ml when the dose of methotrexate is about 15 mg. In another embodiment, the pharmacokinetic profile provides an AUC_{(0-inf)} of about 1979 ng*hr/ml when the dose of methotrexate is about 15 mg. In another embodiment, the pharmacokinetic profile provides a Tₘₐₓ of about 1.25 hours when the dose of methotrexate is about 15 mg. In another embodiment, the pharmacokinetic profile provides a half-life of about 3.68 hours when the dose of methotrexate is about 15 mg. In another embodiment, the pharmacokinetic profile provides a mean Cₘₐₓ of from about 284 ng/ml to about 445 ng/ml when the dose of methotrexate is about 15 mg. In another embodiment, the pharmacokinetic profile provides an AUC₍₀₋ₜ₎ of from about 1556 ng*hr/ml to about 2435 ng*hr/ml when the dose of methotrexate is about 15 mg. In another embodiment, the pharmacokinetic profile provides an AUC₍₀₋₂₄₎ of from about 1558 ng*hr/ml to about 2435 ng*hr/ml when the dose of methotrexate is about 15 mg. In another embodiment, the pharmacokinetic profile provides an AUC_{(0-inf)} of from about 1583 ng*hr/ml to about 2473 ng*hr/ml when the dose of methotrexate is about 15 mg. In another embodiment, the pharmacokinetic profile provides a Tₘₐₓ of from about 1 hour to about 1.56 hours is when the dose of methotrexate is about 15 mg. In another embodiment, the pharmacokinetic profile provides a half-life of from about 2.94 hours to about 4.60 hours when the dose of methotrexate is about 15 mg. In another embodiment, the pharmacokinetic profile provides a mean Cₘₐₓ of about 417 ng/ml when the dose of methotrexate is about 20 mg. In another embodiment, the pharmacokinetic profile provides an AUC₍₀₋ₜ₎ of about 2188 ng*hr/ml when the dose of methotrexate is about 20 mg. In another embodiment, the pharmacokinetic profile provides an AUC₍₀₋₂₄₎ of about 2188 ng*hr/ml when the dose of methotrexate is about 20 mg. In another embodiment, the pharmacokinetic profile provides an AUC_{(0-inf)} of about 2219 ng*hr/ml when the dose of methotrexate is about 20 mg. In another embodiment, the pharmacokinetic profile provides a Tₘₐₓ of about 1.17 hours when the dose of methotrexate is about 20 mg. In another embodiment, the pharmacokinetic profile provides a half-life of about 3.58 hours when the dose of methotrexate is about 20 mg. In another embodiment, the pharmacokinetic profile provides a mean Cₘₐₓ of from about 333 ng/ml to about 521 ng/ml when the dose of methotrexate is about 20 mg. In another embodiment, the pharmacokinetic profile provides an AUC₍₀₋ₜ₎ of from about 1750 ng*hr/ml to about 2735 ng*hr/ml when the dose of methotrexate is about 20 mg. In another embodiment, the pharmacokinetic profile provides an AUC₍₀₋₂₄₎ of from about 1750 ng*hr/ml to about 2735 ng*hr/ml when the dose of methotrexate is about 20 mg. In another embodiment, the pharmacokinetic profile provides an AUC_{(0-inf)} of from about 1775 ng*hr/ml to about 2773 ng*hr/ml when the dose of methotrexate is about 20 mg. In another embodiment, the pharmacokinetic profile provides a Tₘₐₓ of from about 0.93 hours to about 1.46 hours when the dose of methotrexate is about 20 mg. In another embodiment, the pharmacokinetic profile provides a half-life of from about 2.86 hours to about 4.47 hours when the dose of methotrexate is about 20 mg. In another embodiment, the pharmacokinetic profile provides a mean Cₘₐₓ of about 491 ng/ml when the dose of methotrexate is about 25 mg. In another embodiment, the pharmacokinetic profile provides an AUC₍₀₋ₜ₎ of about 2799 ng*hr/ml when the dose of methotrexate is about 25 mg. In another embodiment, the pharmacokinetic profile provides an AUC₍₀₋₂₄₎ of about 2799 ng*hr/ml when the dose of methotrexate is about 25 mg. In another embodiment, the pharmacokinetic profile provides an AUC_{(0-inf)} of about 2836 ng*hr/ml when the dose of methotrexate is about 25 mg. In another embodiment, the pharmacokinetic profile provides a Tₘₐₓ of about 1.23 hours when the dose of methotrexate is about 25 mg. In another embodiment, the pharmacokinetic profile provides a half-life of about 3.78 hours when the dose of methotrexate is about 25 mg. In another embodiment, the pharmacokinetic profile provides a mean Cₘₐₓ of from about 392 ng/ml to about 613 ng/ml when the dose of methotrexate is about 25 mg. In another embodiment, the pharmacokinetic profile provides an AUC₍₀₋ₜ₎ of from about 2239 ng*hr/ml to about 3498 ng*hr/ml. In another embodiment, the pharmacokinetic profile provides an AUC₍₀₋₂₄₎ of from about 2239 ng*hr/ml to about 3498 ng*hr/ml when the dose of methotrexate is about 25 mg. In another embodiment, the pharmacokinetic profile provides an AUC_{(0-inf)} of from about 2268 ng*hr/ml to about 3545 ng*hr/ml when the dose of methotrexate is about 25 mg. In another embodiment, the pharmacokinetic profile provides a Tₘₐₓ of from about 0.98 hours to about 1.54 hours when the dose of methotrexate is about 25 mg. In another embodiment, the pharmacokinetic profile provides a half-life of from about 3.02 hours to about 4.72 hours when the dose of methotrexate is about 25 mg.

In an aspect, the present invention provides a method of treating an autoimmune disorder in a subject in need of treatment, said method comprising introducing into the subcutaneous tissue of said subject, from a needle assisted jet injection device, a composition comprising methotrexate in dose ranging from about 5 mg to about 50 mg, wherein said method provides a pharmacokinetic profile that increases linearly in proportion to increases in methotrexate dose level. In another embodiment, the pharmacokinetic profile provides methotrexate exposure (AUC or Cₘₐₓ) that increases linearly in proportion to increases in methotrexate dose level. In another embodiment, the pharmacokinetic profile provides an AUC that increases linearly in proportion to increases in methotrexate dose level. In another embodiment, the pharmacokinetic profile provides a Cₘₐₓ that increases linearly in proportion to increases in methotrexate dose level.

According to a further feature of the present invention, there is provided a treatment schedule. In one embodiment, said method of treating an autoimmune disorder comprises introducing into the subcutaneous tissue of a subject, a composition comprising methotrexate, wherein said composition contains an entire weekly dose of methotrexate and is administered once a week in a single dose. In an embodiment, an entire weekly dose of said methotrexate is divided into multiple doses and injected as multiple daily injections. In another embodiment, an entire weekly dose of said methotrexate is divided into multiple doses and injected over at least two or more days over a period of a week.

In another aspect, the present invention provides a composition comprising methotrexate for use in the treatment of an autoimmune disorder in a subject in need of treatment by introducing into the subcutaneous tissue of said subject, from a needle assisted jet injection device, the composition comprising methotrexate in a dose ranging from about 5 mg to about 50 mg, wherein the pharmacokinetic profile of said methotrexate, obtained following delivery of said methotrexate by said needle assisted jet injection device, is substantially the same as the pharmacokinetic profile of the same dose of said methotrexate when administered by an intramuscular injection or a subcutaneous injection.

In an embodiment, the composition provides a pharmacokinetic profile comprises a set of one or more pharmacokinetic parameters selected from the group consisting of: (a) bioavailability of said methotrexate following said delivery by said needle assisted jet injection device; (b) time of peak concentration (Tₘₐₓ) of a blood (serum or plasma) concentration-time curve of said methotrexate following said delivery by said needle assisted jet injection device; (c) peak height concentration (Cₘₐₓ) of a blood (or serum or plasma) concentration time curve of said methotrexate following said delivery by said needle assisted jet injection device; (d) area under a blood (serum or plasma) concentration-time curve (AUC) of said methotrexate following delivery by said needle assisted jet injection device; and (e) combinations of (a), (b), (c) and (d).

In an embodiment, the composition said Cₘₐₓ has a value selected from the group consisting of: from about 170 ng/ml to about 266 ng/ml when the dose, of methotrexate is about 10 mg; from about 284 ng/ml to about 445 ng/ml when the dose of methotrexate is about 15 mg; from about 333 ng/ml to about 521 ng/ml when the dose of methotrexate is about 20 mg; and from about 392 ng/ml to about 613 ng/ml when the dose of methotrexate is about 25 mg. In a further embodiment, said Cₘₐₓ has a value selected from the group consisting of: about 213 ng/ml when the dose of methotrexate is about 10 mg; about 356 ng/ml when the dose of methotrexate is about 15 mg; about 417 ng/ml when the dose of methotrexate is about 20 mg; and about 491 ng/ml when the dose of methotrexate is about 25 mg.

In an embodiment, the composition provides an AUC selected from the group consisting of: AUC₍₀₋ₜ₎ of from about 912 ng*hr/ml to about 1426 ng*hr/ml when the dose of methotrexate is about 10 mg; an AUC₍₀₋₂₄₎ of from about 920 ng*hr/ml to about 1437 ng*hr/ml when the dose of methotrexate is about 10 mg; and an AUC_{(0-inf)} is from about 929 ng*hr/ml to about 1451 ng*hr/ml when the dose of methotrexate is about 10 mg; and combinations thereof. In a further embodiment, said AUC is selected from the group consisting of: AUC₍₀₋ₜ₎ of about 1141 ng*hr/ml; AUC₍₀₋₂₄₎ of about 1150 ng*hr/ml; AUC_{(0-inf)} of about 1161 ng*hr/ml; and combinations thereof.

In an embodiment, the composition provides an AUC selected from the group consisting of AUC₍₀₋ₜ₎ of from about 1556 ng*hr/ml to about 2435 ng*hr/ml when the dose of methotrexate is about 15 mg; an AUC₍₀₋₂₄₎ of from about 1558 ng*hr/ml to about 2435 ng*hr/ml when the dose of methotrexate is about 15 mg; an AUC_{(0-inf)} of from about 1583 ng*hr/ml to about 2473 ng*hr/ml when the dose of methotrexate is about 15 mg; and combinations thereof. In a further embodiment, said AUC has a value selected from the group consisting of: AUC₍₀₋ₜ₎ of about 1945 ng*hr/ml; AUC₍₀₋₂₄₎ of about 1948 ng*hr/ml; AUC_{(0-inf)} of about 1979 ng*hr/ml; and combinations thereof.

In an embodiment, the composition provides an AUC selected from the group consisting of: an AUC₍₀₋ₜ₎ of from about 1750 ng*hr/ml to about 2735 ng*hr/ml when the dose of methotrexate is about 20 mg; an AUC₍₀₋₂₄₎ of from about 1750 ng*hr/ml to about 2735 ng*hr/ml when the dose of methotrexate is about 20 mg; an AUC_{(0-inf)} of from about 1775 ng*hr/ml to about 2773 ng*hr/ml when the dose of methotrexate is about 20 mg; and combinations thereof. In a further embodiment, said AUC has a value selected from the group consisting of: AUC₍₀₋ₜ₎ of about 2188 ng*hr/ml; AUG₍₀₋₂₄₎ of about 2188 ng*hr/ml; AUC_{(0-inf)} of about 2219 ng*hr/ml; and combinations thereof.

In an embodiment, the composition provides an AUC selected from the group consisting of: an AUC₍₀₋ₜ₎ of from about 2239 ng*hr/ml to about 3498 ng*hr/ml when the dose of methotrexate is about 25 mg; an AUC₍₀₋₂₄₎ of from about 2239 ng*hr/ml to about 3498 ng*hr/ml when the dose of methotrexate is about 25 mg; and an AUC₍₀₄₀ of from about 2268 ng*hr/ml to about 3545 ng*hr/ml when the dose of methotrexate is about 25 mg. In a further embodiment, said AUC has a value selected from the group consisting of: AUC₍₀₋ₜ₎ of about 2799 ng*hr/ml; an AUC₍₀₋₂₄₎ of about 2799 ng*hr/ml; an AUC_{(0-inf)} of about 2836 ng*hr/ml; and combinations thereof.

In an embodiment, the composition provides a set of one or more pharmacokinetic parameters selected from the group consisting of: a Tₘₐₓ of from about 1.06 hours to about 1.66 hours when the dose of methotrexate is about 10 mg; a half-life of from about 2.6 hours to about 4.06 hours when the dose of methotrexate is about 10 mg; and a combination thereof. In a further embodiment, said set of one or more pharmacokinetic parameters is selected from the group consisting of: a Tₘₐₓ of about 1.33 hours; a half-life of about 3 hours.

In an embodiment, the composition provides a set of one or more pharmacokinetic selected from the group consisting of: a Tₘₐₓ of from about 1 hour to about 1.56 hours is when the dose of methotrexate is about 15 mg; a half-life of from about 2.94 hours to about 4.60 hours when the dose of methotrexate is about 15 mg; and a combination thereof. In a further embodiment, said set of one or more pharmacokinetic parameters is selected from the group consisting of: a Tₘₐₓ of about 1.25 hours; a half-life of about 3.68 hours; and combinations thereof.

In an embodiment, the composition provides a set of one or more pharmacokinetic parameters selected from the group consisting of: a Tₘₐₓ of from about 0.93 hours to about 1.46 hours when the dose of methotrexate is about 20 mg; a half-life of from about 2.86 hours to about 4.47 hours when the dose of methotrexate is about 20 mg; and a combination thereof. In a further embodiment, said Tₘₐₓ is about 1.17 hours; said half-life is about 3.58 hours.

In an embodiment, the composition provides a set of one or more pharmacokinetic parameters selected from the group consisting of: a Tₘₐₓ of from about 0.98 hours to about 1.54 hours when the dose of methotrexate is about 25 mg; a half-life of from about 3.02 hours to about 4.72 hours when the dose of methotrexate is about 25 mg. In a further embodiment, said Tₘₐₓ is about 1.23 hours; said half-life is about 3.78 hours.

In an embodiment of the composition, said methotrexate is present in an amount ranging from about 5 mg to about 10 mg, from about 5 mg to about 15 mg, from about 5 mg to about 20 mg, from about 5 mg to about 25 mg, from about 5 mg to about 30 mg, from about 5 mg to about 40 mg, from about 5 mg to 50 mg, 7.5 mg to about 10 mg, from about 7.5 mg to about 15 mg, from about 7.5 mg to about 20 mg, from about 7.5 mg to about 25 mg, from about 7.5 mg to about 30 mg, from about 7.5 mg to about 40 mg, from about 7.5 mg to 50 mg, from 10 mg to about 15 mg, from about 10 mg to about 20 mg, from about 10 mg to about 25 mg, from about 10 to about 30 mg, from about 10 mg to about 40 mg, from about 15 mg to about 20 mg, from about 15 mg to about 25 mg, from about 15 to about 30 mg, from about 15 to about 35 mg, from about 15 mg to about 40 mg, from about 15 mg to about 35 mg, from about 15 mg to about 50 mg, from about 20 mg to about 25 mg, from about 20 to about 30 mg, from about 20 to about 35 mg, from about 20 mg to about 40 mg, from about 20 mg to about 50 mg, from about 25 to about 30 mg, from about 25 to about 35 mg, from about 25 mg to about 40 mg, from about 25 mg to about 50 mg, from about 30 to about 35 mg, from about 30 mg to about 40 mg, from about 30 mg to about 50 mg, or from about 35 mg to about 50 mg.

In another embodiment of the composition, said autoimmune disorder is selected from the group consisting of Juvenile idiopathic arthritis (JIA), Juvenile rheumatoid arthritis (JRA), Psoriatic arthritis (PA), and Rheumatoid arthritis (RA).

In yet another embodiment of the composition, said methotrexate is administered in combination with a set of one or more biologics. In a further embodiment, said set of one or more biologics comprises one or more alpha TNFs inhibitors. In one embodiment, said set of one or more biologics comprises Etanercept (or Enbrel) or intliximab (or Remicade) or a combination thereof.

In an embodiment of the composition, said use provides a pharmacokinetic profile that increases linearly in proportion to increases in methotrexate dose level.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention can be embodied in different forms and thus should not be construed as being limited to the embodiments set forth herein.

FIG. 1A illustrates dose-normalized methotrexate concentration (ng/ml/mg) versus time on original scale by treatment;

FIG. 1B illustrates a plot of geometric mean of dose-normalized methotrexate concentration (ng/ml/mg) versus time on logarithmic scale by treatment.

FIG. 2 illustrates a summary of methotrexate (10 mg, 15 mg, and 20 mg) pharmacokinetic parameters following subcutaneous administration utilizing Vibex™ device;

FIG. 3 illustrates a summary of methotrexate pharmacokinetic parameters by Treatment Group (following subcutaneous administration of methotrexate (25 mg) utilizing Vibex™ device and subcutaneous administration of methotrexate (10 mg) utilizing needle and syringe);

FIG. 4 illustrates a summary of methotrexate (15 mg, 20 mg, and 25 mg) pharmacokinetic parameters following subcutaneous administration utilizing needle and syringe;

FIG. 5 illustrates a summary of methotrexate (10 mg, 15 mg, and 20 mg) pharmacokinetic parameters following intramuscular administration utilizing needle and syringe;

FIG. 6 illustrates a summary of methotrexate (25 mg) pharmacokinetic parameters following intramuscular administration utilizing needle and syringe;

FIG. 7 illustrates a summary of dose-normalized methotrexate pharmacokinetic parameters by Treatment Group (Vibex™ device SC treatment group and needle and syringe SC group);

FIG. 8 illustrates a summary of dose-normalized methotrexate pharmacokinetic parameters by Treatment Group (syringe IM group);

FIG. 9 illustrates mixed-model analysis of dose-normalized methotrexate pharmacokinetic parameters by Treatment Group (Vibex™ device SC treatment group and needle and syringe SC group);

FIG. 10 illustrates mixed-model analysis of dose-normalized methotrexate pharmacokinetic parameters by Treatment Group (Vibex™ device SC treatment group and needle and syringe IM group);

FIG. 11 illustrates geometric mean Cₘₐₓ(ng/ml) versus methotrexate (MTX) dose (mg) by treatment Group Treatment Group (A: Vibex™ device SC treatment group; B: needle and syringe SC treatment group; C: needle and syringe IM treatment group); and

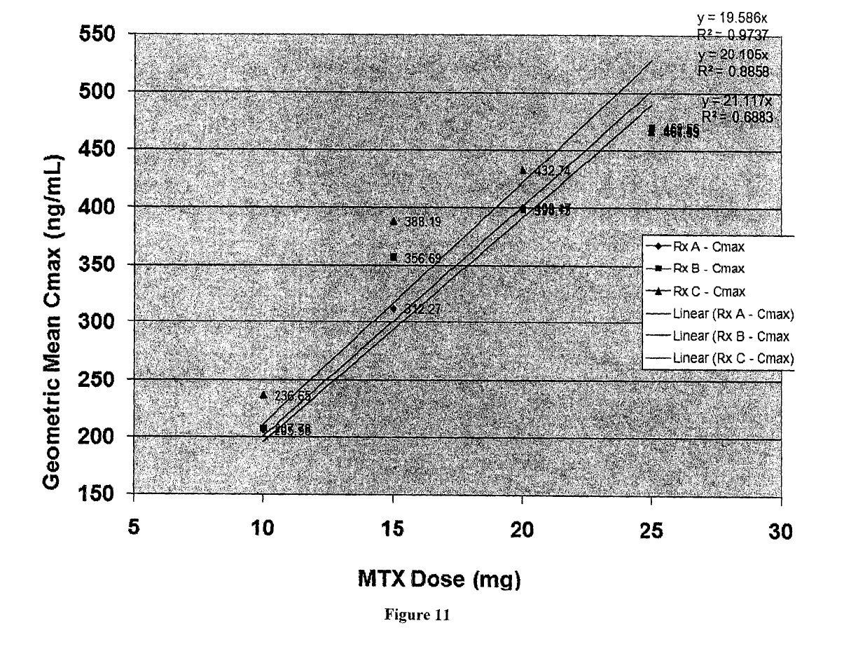

FIG. 12 illustrates geometric mean AUC (ng*hr/ml) versus methotrexate (MTX) dose (mg) by treatment Group Treatment Group (A: Vibex™ device SC treatment group; B: needle and syringe SC treatment group; C: needle and syringe IM treatment group).

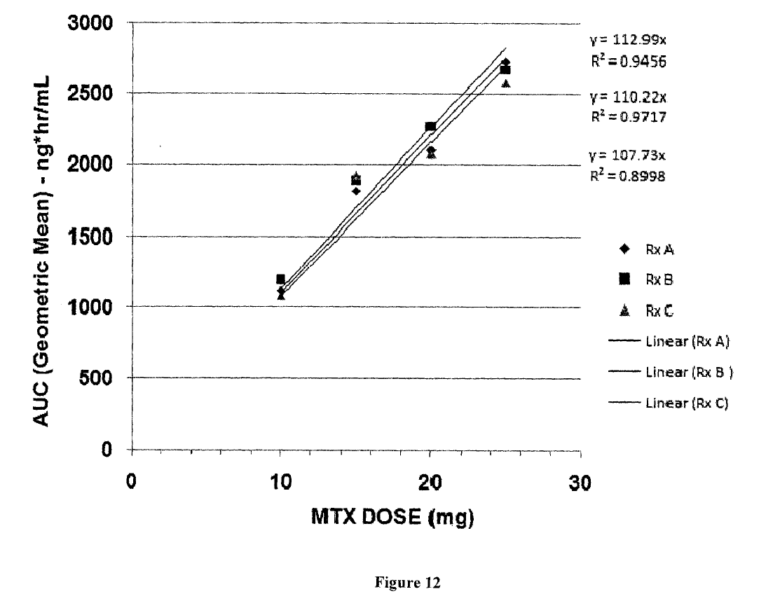

### DETAILED DESCRIPTION OF THE INVENTION

The present subject matter will now be described more fully hereinafter with reference to the accompanying Figures and Examples, in which representative embodiments are shown. The present subject matter can, however, be embodied in different forms and should not be construed as limited to the embodiments set forth herein. Rather, these embodiments are provided to describe and enable one of skill in the art. Unless otherwise defined, all technical and scientific terms used herein have the same meaning as commonly understood by one of ordinary skill in the art to which the subject matter pertains. All publications, patent applications, patents, and other references mentioned herein are incorporated by reference in their entirety.

The pharmacokinetic profile of methotrexate is generally known (see, e.g., Aquerreta, I., et al., Ped. Blood & Cancer (2003); 42(1), 52-58; and Seideman, P., et al., Br. J. Clin. Pharmacol. (1993) April; 35(4): 409-412). Methotrexate is a weak dicarboxylic acid with an acid association constant of about 4.8 to about 5.5, and thus exists mostly in its ionized state at physiologic pH. After intravenous administration, the initial average distribution volume of methotrexate is typically about 0.18 L/Kg (or about 18% of the subject's body weight) and the average steady-state distribution volume typically ranges from about 0.4 L/Kg to about 0.8 L/Kg (or about 40% to about 80% of the subject's body weight). Methotrexate is generally completely absorbed from parenteral routes of injection. After intramuscular injection of methotrexate, peak serum concentrations (Cₘₐₓ) occur in about 30 to about 60 minutes (Tₘₐₓ) in most patients. However, individual plasma concentrations of injected methotrexate have been reported to vary widely between individual subjects. For example, in pediatric patients with juvenile rheumatoid arthritis, the average mean serum concentrations of methotrexate were about 0.59 μM (averaged over a range of about 0.03 μM to about 1.40 μM) at about 1 hour, an average of about 0.44 μM (averaged over a range of about 0.01 μM to about 1.00 μM) at about 2 hours, and an average of about 0.29 μM (averaged over a range of about 0.06 μM to about 0.58 μM) at about 3 hours. In pediatric patients receiving methotrexate injections for acute lymphocytic leukemia (at doses of about 6.3 mg/m² to about 30 mg/m²) or for juvenile rheumatoid arthritis (at doses of about 3.75 mg/m² to about 26.2 mg/m²), the terminal half-life of methotrexate has been reported to range from about 0.7 hours to about 5.8 hours, or from 0.9 hours to about 2.3 hours, respectively.

### I. DEFINITIONS

“AUC” is the area under a curve representing the concentration of a compound, such as methotrexate as defined herein, or metabolite thereof in the blood or plasma or serum of a patient as a function of time following administration of the compound to the patient. For example, following administration of methotrexate as described herein, the AUC of methotrexate may be determined by measuring the concentration of it or its metabolite in blood using methods such as liquid chromatography-tandem mass spectrometry (LC-MS/MS), at various time intervals, and calculating the area under the blood, plasma or serum concentration-versus-time curve. The concentration versus time curve is sometime referred to as the pharmacokinetic profile. Suitable methods for calculating the AUC from a drug concentration-versus-time curve are well known in the art. Therefore, an AUC for methotrexate may be determined by measuring the concentration of methotrexate in the blood of a patient following administration of methotrexate to a patient. AUC₍₀₋₂₄₎ is the area under the curve from administration (time 0) to 24 hours following administration. AUC_{(ss,24)} is the area under the curve over a 24 hour period following a dosing regimen administered over a period of days (steady state). AUC₍₀₋ₜ₎ is the area under the concentration versus time curve from the time of dosing methotrexate to the last measurable concentration of methotrexate.

“Bioavailability” refers to the amount of a compound, such as methotrexate, that reaches the systemic circulation of a patient following administration of the compound to the patient and can be determined by evaluating, for example, the blood or plasma concentration for the compound.

“Patient” and “Subject” both independently include mammals, such as for example, humans.

“Pharmaceutically acceptable” refers to approved or approvable by a regulatory agency of a federal or a state government, listed in the U.S. Pharmacopeia, or listed in other generally recognized pharmacopeia for use in mammals, including humans.

“Pharmaceutically acceptable salt” refers to a salt of a compound, such as methotrexate sodium, that is pharmaceutically acceptable and that possesses the desired pharmacological activity of the parent compound. Such salts include (a) acid addition salts that are formed with inorganic acids, including hydrochloric acid, hydrobromic acid, sulfuric acid, nitric acid, phosphoric acid; or that are formed with organic acids, including acetic acid, propionic acid, hexanoic acid, cyclopentapropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptnoic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid; and (b) salts formed when an acidic proton present in the parent compound either is replaced by a metal ion, e.g., an alkali metal ion, an alkaline earth metal ion, or an aluminum ion; or coordinates with an organic base, including ethanolamine, diethanolamine, triethanolamine, N-methylglucamine. In certain embodiments, the salt of methotrexate is the hydrochloride salt. In other embodiments, the salt of methotrexate is the sodium salt.

As used herein the term “about” means plus or minus 10% of the value referenced.

### II. METHODS OF TREATING AUTOIMMUNE DISORDERS

The concentration of methotrexate in the blood stream of a subject will depend on the amount of methotrexate in the composition administered to the subject as well as the route of administration and the specific formulation used. It is well known in the art that even though IM and SC administration of methotrexate results in similar total exposure in terms of AUC, an increase in Cₘₐₓ of from 15% to 20% is observed with methotrexate dosed at higher strength than 10 mg. In particular, it has been reported that Metoject® 50 mg/ml (a ready to use pre-filled syringe containing methotrexate and an injection needle), results in similar total exposure in terms of AUC, but provides Cₘₐₓ that is 15% to 20% higher when compared with the lower strength Metoject® 10 mg/ml. Therefore, it will be apparent to one of skill in the art that the absolute and relative amount of methotrexate, or pharmaceutically acceptable salts thereof, administered to a subject via subcutaneous and intramuscular administration would result in different pharmacokinetic profiles and therefore dissimilar or different pharmacokinetic parameters estimated based on the respective pharmacokinetic profiles.

Contrary to the predicted pharmacokinetics of methotrexate, the present inventors have discovered that introducing into the subcutaneous tissue of a subject from a needle assisted jet injection device, such as a Vibex™ device, methotrexate, in dose ranging from about 5 mg to about 50 mg, provide methotrexate pharmacokinetic profile that is substantially the same (or similar) as the pharmacokinetic profile of the same dose of methotrexate when administered to the subject with a needle/syringe, intramuscularly, or a subcutaneously.

Accordingly, the present disclosure provides, in part, a method of treating an autoimmune disease with methotrexate and/or a pharmaceutically acceptable salt thereof. In an exemplary embodiment, the method surprisingly and advantageously provides a pharmacokinetic profile for methotrexate dose ranging from about 5 mg to about 50 mg that is substantially the same or similar to the pharmacokinetic profile of the same dose of methotrexate when administered with needle and syringe, intramuscularly or subcutaneously.

In an embodiment, methotrexate administered to a subject in accordance with the methods of the invention provides pharmacokinetics, including systemic bioavailability, that is substantially the same as pharmacokinetics, including systemic bioavailability, of methotrexate when the same dose of methotrexate is administered to said subject using an intramuscular injection and/or a subcutaneous injection. In another embodiment, the method of treating an autoimmune disorder in accordance with the invention, comprises introducing into the subcutaneous tissue of a subject, from a needle assisted jet injection device, a composition comprising methotrexate in a dose ranging from about 5 mg to about 50 mg, wherein the pharmacokinetic profile of said methotrexate delivered by said needle assisted jet injection device is substantially the same as the pharmacokinetic profile of the same dose of said methotrexate when administered to said subject by an intramuscular injection or a subcutaneous injection.

In an embodiment, methotrexate administered in accordance with the invention achieves comparable pharmacokinetic profile by generating Cₘₐₓ and Tₘₐₓ for the same period of time as compared to when the same dose of methotrexate is delivered via an intramuscular or subcutaneous route.

In some embodiment, a method of treating an autoimmune disorder in accordance with the invention comprises introducing through the subject's skin and into the subcutaneous tissue of said subject a composition comprising methotrexate, wherein said methotrexate is in a dose ranging from about 5 mg to about 50 mg, wherein said methotrexate has a substantially the same pharmacokinetic profile when introduced into the subcutaneous tissue of the subject using an a needle assisted jet injection device, or using an intramuscular injection or a subcutaneous injection. In an embodiment, said methotrexate is present in an amount ranging from about 5 mg to about 7.5 mg, from about 5 mg to about 10 mg, from about 5 mg to about 12.5 mg, from 5 mg to about 15 mg, from about 5 mg to about 17.5 mg, from about 5 mg to about 20 mg, from about 5 mg to about 22.5 mg, from about 5 mg to about 25 mg, from about 5 to about 30 mg, from about 5 mg to about 40 mg, or from about 5 mg to about 50 mg, from about 7.5 mg to about 10 mg, from about 7.5 mg to about 12.5 mg, from about 7.5 mg to about 15 mg, from about 7.5 mg to about 17.5 mg, from about 7.5 mg to about 20 mg, from about 7.5 mg to about 22.5 mg, from about 7.5 mg to about 25 mg, from about 7.5 mg to about 30 mg, from about 7.5 mg to about 35 mg, from about 7.5 mg to about 40 mg, from about 7.5 mg to about 45 mg, from about 7.5 mg to about 50 mg, from about 10 mg to about 12.5 mg, from about 10 mg to about 15 mg, from about 10 mg to about 17.5 mg, from about 10 mg to about 20 mg, from about 10 mg to about 22.5 mg, from about 10 mg to about 25 mg, from about 10 to about 30 mg, from about 10 mg to about 40 mg, or from about 10 mg to about 50 mg. In another embodiment, said methotrexate is present in said in an amount ranging from about 15 mg to about 17.5 mg, from about 15 mg to about 20 mg, from about 10 mg to about 22.5 mg, from about 15 mg to about 25 mg, from about 15 to about 30 mg, from about 15 mg to about 35 mg, from about 15 mg to about 40 mg, or from about 15 mg to about 50 mg. In yet another embodiment, said methotrexate is present in an amount ranging from about 20 mg to about 22.5 mg, from about 20 mg to about 25 mg, from about 20 mg to about 30 mg, from about 20 to about 35 mg, from about 20 mg to about 40 mg, or from about 35 mg to about 50 mg. In another embodiment, said methotrexate is present in an amount ranging from about 22.5 mg to about 30 mg, from about 22.5 mg to about 35 mg, from about 22.5 mg to about 40 mg, from about 22.5 to about 50 mg, from about 25 mg to about 30 mg, from about 25 mg to about 35 mg, from about 25 mg to about 40 mg, or from about 25 to about 50 mg or higher.

### III. PHARMACOKINETICS

In one embodiment of a 10 mg dose of the present invention, the pharmacokinetic profile provides a mean Cₘₐₓ of about 213 ng/ml. In another embodiment, the pharmacokinetic profile provides an AUC₍₀₋ₜ₎ of about 1141 ng*hr/ml. In another embodiment, the pharmacokinetic profile provides an AUC₍₀₋₂₄₎ of about 1150 ng*hr/ml. In another embodiment, the pharmacokinetic profile provides an AUC_{(0-inf)} of about 1161 ng*hr/ml. In another embodiment, the pharmacokinetic profile provides a Tₘₐₓ of about 1.33 hours. In another embodiment, the pharmacokinetic profile provides a half-life of about 3 hours.

In another embodiment of a 10 mg dose of the present invention, the pharmacokinetic profile provides a mean Cₘₐₓ of from about 170 ng/ml to about 266 ng/ml. In another embodiment, the pharmacokinetic profile provides an AUC₍₀₋ₜ₎ of from about 912 ng*hr/ml to about 1426 ng*hr/ml.

In another embodiment, the pharmacokinetic profile provides an AUC₍₀₋₂₄₎ of from about 920 ng*hr/ml to about 1437 ng*hr/ml. In another embodiment, the pharmacokinetic profile provides an AUC_{(0-inf)} is from about 929 ng*hr/ml to about 1451 ng*hr/ml. In another embodiment, the pharmacokinetic profile provides a Tₘₐₓ of from about 1.06 hours to about 1.66 hours. In another embodiment, the pharmacokinetic profile provides a half-life of from about 2.6 hours to about 4.06 hours.

In an embodiment of a 15 mg dose of the present invention, the pharmacokinetic profile provides a mean Cₘₐₓ of about 356 ng/ml. In another embodiment, the pharmacokinetic profile provides an AUC₍₀₋ₜ₎ of about 1945 ng*hr/ml. In another embodiment, the pharmacokinetic profile provides an AUC₍₀₋₂₄₎ of about 1948 ng*hr/ml. In another embodiment, the pharmacokinetic profile provides an AUC_{(0-inf)} of about 1979 ng*hr/ml. In another embodiment, the pharmacokinetic profile provides a Tₘₐₓ of about 1.25 hours. In another embodiment, the pharmacokinetic profile provides a half-life of about 3.68 hours.

In another embodiment of a 15 mg dose of the present invention, the pharmacokinetic profile provides a mean Cₘₐₓ of from about 284 ng/ml to about 445 ng/ml. In another embodiment, the pharmacokinetic profile provides an AUC₍₀₋ₜ₎ of from about 1556 ng*hr/ml to about 2435 ng*hr/ml. In another embodiment, the pharmacokinetic profile provides an AUC₍₀₋₂₄₎ of from about 1558 ng*hr/ml to about 2435 ng*hr/ml. In another embodiment, the pharmacokinetic profile provides an AUC_{(0-inf)} of from about 1583 ng*hr/ml to about 2473 ng*hr/ml. In another embodiment, the pharmacokinetic profile provides a Tₘₐₓ of from about 1 hour to about 1.56 hours. In another embodiment, the pharmacokinetic profile provides a half-life of from about 2.94 hours to about 4.60 hours.

In an embodiment of a 20 mg dose of the present invention, the pharmacokinetic profile provides a mean Cₘₐₓ of about 417 ng/ml. In another embodiment, the pharmacokinetic profile provides an AUC₍₀₋ₜ₎ of about 2188 ng*hr/ml. In another embodiment, the pharmacokinetic profile provides an AUC₍₀₋₂₄₎ of about 2188 ng*hr/ml. In another embodiment, the pharmacokinetic profile provides an AUC_{(0-inf)} of about 2219 ng*hr/ml. In another embodiment, the pharmacokinetic profile provides a Tₘₐₓ of about 1.17 hours. In another embodiment, the pharmacokinetic profile provides a half-life of about 3.58 hours.

In another embodiment of a 20 mg dose of the present invention, the pharmacokinetic profile provides a mean Cₘₐₓ of from about 333 ng/ml to about 521 ng/ml. In another embodiment, the pharmacokinetic profile provides an AUC₍₀₋ₜ₎ of from about 1750 ng*hr/ml to about 2735 ng*hr/ml. In another embodiment, the pharmacokinetic profile provides an AUC₍₀₋₂₄₎ of from about 1750 ng*hr/ml to about 2735 ng*hr/ml. In another embodiment, the pharmacokinetic profile provides an AUC_{(0-inf)} of from about 1775 ng*hr/ml to about 2773 ng*hr/ml. In another embodiment, the pharmacokinetic profile provides a Tₘₐₓ of from about 0.93 hours to about 1.46 hours. In another embodiment, the pharmacokinetic profile provides a half-life of from about 2.86 hours to about 4.47 hours.

In an embodiment of 25 mg dose of the present invention, the pharmacokinetic profile provides a mean Cₘₐₓ of about 491 ng/ml. In another embodiment, the pharmacokinetic profile provides an AUC₍₀₋ₜ₎ of about 2799 ng*hr/ml. In another embodiment, the pharmacokinetic profile provides an AUC₍₀₋₂₄₎ of about 2799 ng*hr/ml. In another embodiment, the pharmacokinetic profile provides an AUC_{(0-inf)} of about 2836 ng*hr/ml. In another embodiment, the pharmacokinetic profile provides a Tₘₐₓ of about 1.23 hours. In another embodiment, the pharmacokinetic profile provides a half-life of about 3.78 hours.

In another embodiment of a 25 mg of the present invention, the pharmacokinetic profile provides a mean Cₘₐₓ of from about 392 ng/ml to about 613 ng/ml. In another embodiment, the pharmacokinetic profile provides an AUC₍₀₋ₜ₎ of from about 2239 ng*hr/ml to about 3498 ng*hr/ml.

In another embodiment of 25 mg dose of the present invention, the pharmacokinetic profile provides an AUC₍₀₋₂₄₎ of from about 2239 ng*hr/ml to about 3498 ng*hr/ml. In another embodiment, the pharmacokinetic profile provides an AUC_{(0-inf)} of from about 2268 ng*hr/ml to about 3545 ng*hr/ml. In another embodiment, the pharmacokinetic profile provides a Tₘₐₓ of from about 0.98 hours to about 1.54 hours. In another embodiment, the pharmacokinetic profile provides a half-life of from about 3.02 hours to about 4.72 hours.

In an embodiment of a 5 mg to 50 mg dose of the present invention, the pharmacokinetic profile provides a linear increase in methotrexate exposure with increases in dose of methotrexate administered. In an embodiment, the pharmacokinetic profile provides dose proportional increases in methotrexate exposure (AUC and/or Cₘₐₓ). In another embodiment, the pharmacokinetic profile provides a linear relationship between AUC (ng*h/ml) of methotrexate and dose of methotrexate when the AUC (ng*h/ml) values are plotted against the corresponding dose values in a Cartesian Plane. In another embodiment, the pharmacokinetic profile provides a linear relationship between Cₘₐₓ of methotrexate and dose of methotrexate when the Cₘₐₓ values are plotted against the corresponding dose values in Cartesian Plane.

In an embodiment of 10 mg, 15 mg, 20 mg and 25 mg of the present, the pharmacokinetic profile provides an AUC that increases linearly in proportion to dose strength. In an embodiment of 10 mg, 15 mg, 20 mg and 25 mg of the present, the pharmacokinetic profile provides a Cₘₐₓ that increases linearly in proportion to dose strength.

Without wishing to be bound by theory, based on the dose proportionality observed for the dose range of 10 mg to 25 mg, it is believed that with the methods of the present invention lower doses, including 5 mg and 7.5 mg, and higher doses, including 30 mg, 35 mg, 40 mg, 45 mg and 50 mg or higher, would show the dose proportionality (e.g. linearity between AUC and/or Cₘₐₓ and dose).

Accordingly, another embodiment of the present invention provides a method of treating an autoimmune disorder in a subject in need of treatment, said method comprising introducing into the subcutaneous tissue of said subject, from a needle assisted jet injection device, a composition comprising methotrexate in a dose ranging from about 5 mg to about 50 mg, wherein said method provides a pharmacokinetic profile whereby methotrexate exposure increases linearly in proportion to increases in the dose strength (or level) of methotrexate. In an embodiment, the pharmacokinetic profile provides an AUC that increases linearly in proportion to increases in the dose strength (or level) of methotrexate administered. In another embodiment, the pharmacokinetic profile provides a Cₘₐₓ that increases linearly in proportion to increases in methotrexate dose level administered.

Generally, the method of the present invention can be used to treat any suitable autoimmune disorder. Examples of autoimmune disorders suitable for treatment with the method of the present invention include, without limitations, Sarcoidosis; Antineutrophil cystoplasmic antibodies (ANCA)-associated vasculitides; Large vessel vasculitis, including giant cell arteritis, Takayasu arteritis, and polymyalgia rheumatic; Adult-onset Still's disease; Inflammatory myopathies, including dermatomyositis and polymyositis; Scleroderma and mixed connective tissue disease; Systemic lupus erythematosus; Inflammatory bowel diseases, including Crohn's disease; Uveitis; Psoriasis; Psoriatic arthritis (PsA); and combinations thereof.

### IV. POWERED INJECTORS/NEEDLE ASSISTED JET INJECTORS

By using powered injectors and needle-assisted jet injection devices of the present invention, methotrexate may be injected into a subject more precisely and completely than if it were injected via a manual syringe, and in less than about 5 seconds, in less than about 4 seconds, in less than about 3 seconds, in less than about 2 seconds or in less than about 1 seconds. Examples of power injectors, including power jet injectors, and needle assisted jet injectors that are suitable for use with the methods of the present inventions can be found in International Patent Application No. PCT/US2010/028011 (“the '011 application”, now published as WO 2010/108116 A1), filed 19 Mar. 2010, which is entitled “Hazardous Agent Injection System” and claims priority benefit from U.S. Provisional Patent Application No. 61/162,114, filed Mar. 20, 2009, all of which are incorporated herein by reference in their entirety.

It is believed that methotrexate when administered via a powered injector in accordance with the present invention will enhance patient compliance by allowing non-clinical administration of methotrexate via self-administration, as compared to requiring the patient to obtain injections from a medical professional, as compared to oral dosage forms which may require administration up to several times per week, a regimen that is inconvenient for patients and difficult for patients to remember. Compliance may be further enhanced by the speed at which the powered injector and/or needle assisted jet injector delivers methotrexate into an injection site (e.g., subcutaneous tissue) is less than 5 seconds. In some embodiment, said powered injector and/or needle assisted jet injector delivers methotrexate into an injection site in less than about 4 seconds, in less than about 3 seconds, in less than about 2 seconds or in less than about 1 seconds. Additionally, it is believed that a power injector and/or needle assisted jet injector in accordance with the present invention is capable of delivering methotrexate more precisely, in a controlled manner of delivery, thereby reducing the exposure of methotrexate to the outside of the injection site and, in some embodiments, eliminating that exposure completely. In some embodiments, the powered injector or jet injector or powered jet injector is pre-filled with methotrexate so that the user is not required to draw up methotrexate, as they would otherwise be required to do when using a hand-driven, or traditional, syringe. This facilitates operation and accurate dosing in the administration of methotrexate, especially for those patients who have a disease or disorder that makes it difficult for them to draw up medicine and self-inject. It is therefore believed that administration of methotrexate via a powered injector or a powered jet injection device or a needle jet injector will provide a safer means of delivery and will significantly reduce the risk of exposure to methotrexate to non-users of the powered injector or the powered jet injection device or the needle jet injector and reduce the risk of unnecessary toxicity to the patient utilizing the powered injector or the powered jet injection device or the needle jet injector.

Patients treated with methotrexate for some autoimmune disorders, including rheumatoid arthritis, for example, often improve but continue to have active disease. Therefore, in some cases, including the case of patients who have persistent rheumatoid arthritis despite receiving methotrexate, it may be necessary to combine methotrexate with one or more additional therapeutic agents to provide additional benefits to patients, including decreased disease activity, and/or increased functional activity, and/or improved health-related quality of life. Accordingly, an aspect of the present invention provides a method of treating an autoimmune disorder in a subject in need of treatment, comprising introducing into the subcutaneous tissue of said subject, from a needle assisted jet injection device, a composition comprising a combination of methotrexate with one or more therapeutic agents, including tumor necrosis factor (TNF) blockers such as, Etanercept (or Enbrel) and infliximab (or Remicade), wherein said methotrexate is in dose ranging from about 5 mg to about 50 mg, and wherein the pharmacokinetic profile of said methotrexate delivered by said needle assisted jet injection device is substantially the same as the pharmacokinetic profile of the same dose of said methotrexate when administered to said subject by an intramuscular injection or a subcutaneous injection. In one embodiment, said one or more therapeutic agents comprise one or more biologics. In one embodiment, said one or more therapeutic agents comprise one or more tumor necrosis factor (TNF) antagonists (or inhibitors). In an embodiment, said TNF antagonist is the soluble TNF receptor fusion protein (p75) Enbrel.

In an embodiment a powered injector or a powered jet injector for use in accordance with the present invention, uses an energy source that produces moderate to low pressure in the medicament chamber so that a medicament contained in the medicament chamber is fired at a slow speed, similar to the pressure and speed from a finger driven syringe. In another embodiment, said powered injector or said powered jet injector can be configured to have an energy source selected to produce a high pressure in the medicament chamber to eject the medicament with sufficient pressure, force and speed to exit the injector as a fluid jet. Medicament delivered via a high pressure powered injector or a high pressure-powered jet injector is sprayed rapidly into the tissue, and in part remotely from the needle tip, and typically does not deposit the medicament in a bolus local to the needle tip. In an embodiment, a needle assisted jet injector can use lower pressures than a needle free jet injector because it employs a needle to break through the outer part of the skin, but has pressures and speeds that are sufficiently high so that the medicament exits the needle tip as a fluid jet such that leakage typical of shallow needle insertion injections is minimized or does not occur.

In some embodiments of needle-assisted jet injectors or powered injectors or powered jet injection devices for use in accordance with the methods of the invention, injection rates are below about 0.75 ml/sec., in some embodiments below about 0.6 ml/sec., in some embodiments at least about 0.2 ml/sec., in some embodiments at least about 0.3 ml/sec., and in some embodiments at least about 0.4 ml/sec. In some embodiments, the injection rate is selected from below about 0.75 ml/sec, below about 0.7 ml/sec, below about 0.65 ml/sec, below about 0.6 ml/sec, below about 0.55 ml/sec, below about 0.5 ml/sec, below about 0.45 ml/sec, below about 0.4 ml/sec, below about 0.35 ml/sec, below about 0.3 ml/sec, and below about 0.25 ml/sec. In some embodiments, the injection rate is selected from at least about 0.2 ml/sec, at least about 0.25 ml/sec, at least about 0.3 ml/sec, at least about 0.35 ml/sec, at least about 0.4 ml/sec, at least about 0.45 ml/sec, at least about 0.5 ml/sec, at least about 0.55 ml/sec, at least about 0.6 ml/sec, at least about 0.65 ml/sec, and at least about 0.7 ml/sec.

In some embodiments, the injection of the entire amount of medicament is completed in less than about 5 seconds, in some embodiments in less than about 4.5 seconds, in some embodiments in less than about 4 seconds, in some embodiments in less than about 3.5 seconds, in some embodiments in less than about 3 seconds, in some embodiments in less than about 2.5 seconds, in some embodiments in less than about 2 seconds, and in some embodiments in less than about 1.5 seconds. In some embodiments, the medicament injection takes at least about 1 second, in some embodiments at least about 1.5 seconds, in some embodiments at least about 1.75 seconds, in some embodiments at least about 2 seconds, in some embodiments at least about 2.5 seconds, in some embodiments at least about 3 seconds, in some embodiments at least about 3.5 seconds, in some embodiments at least about 4 seconds, and in some embodiments at least about 4.5 seconds. In some embodiments, injection of the medicament occurs at about 0.5 ml/sec., completing an injection of 1 ml in about 1 second. In some embodiments, injection of 0.5 ml of medicament occurs in less than about 1 second. In some embodiments, injection of 1.0 ml of medicament occurs in less than about 2 seconds. In some embodiments, injection of 0.4 ml of medicament occurs in less than about 2 seconds. In some embodiments, injection of 0.4 ml of medicament occurs in less than about 1 second.

In an embodiment, the delivery volume (also injection volume or volume of injection) of methotrexate solution for use in the methods of the invention is less than about 1 ml but greater than 0 ml. In another embodiment, the delivery volume of methotrexate solution is about 0.8 ml. In an embodiment, the delivery volume of methotrexate solution is about 0.7 ml. In another embodiment, the delivery volume of methotrexate solution is about 0.6 ml. In yet another embodiment, the delivery volume of methotrexate solution is about 0.5 ml, about 0.4 ml, or about 0.3 ml.

In an embodiment, the delivery volume (volume of injection or injection volume) of methotrexate of the present is less than about 1 ml but greater than 0 ml. In another embodiment, the delivery volume of methotrexate solution is about 0.8 ml. In an embodiment, the delivery volume of methotrexate solution is about 0.7 ml. In another embodiment, the delivery volume of methotrexate solution is about 0.6 ml. In yet another embodiment, the delivery volume of methotrexate solution is about 0.5 ml, about 0.4 ml, or about 0.3 ml.

In an embodiment, the delivery volume of methotrexate solution is from about 0.2 ml to about 1 ml, from about 0.2 ml to about 0.8 ml, from about 0.2 ml to about 0.7 ml, from about 0.2 ml to about 0.6 ml, from about 0.2 ml to about 0.5 ml, from about 0.2 ml to about 0.4 ml, or from 0.2 ml to about 0.3 ml.

In an embodiment, the delivery volume of methotrexate solution is advantageously held constant at about 1 ml, while the strength of methotrexate is varied from about 5 mg to about 50 mg (e.g., 5 mg/l ml, 6 mg/l ml, 7.5 mg/l ml, 10 mg/l ml; 15 mg/l ml; 20 mg/l ml; 25 mg/l ml; 40 mg/l ml; and 50 mg/l ml). In another embodiment, the delivery volume of methotrexate solution is advantageously held constant at about 0.8 ml, while the strength of methotrexate is varied from about 10 mg to about 50 mg (e.g., 10 mg/0.8 ml; 15 mg/0.8 ml; 20 mg/0.8 ml; 25 mg/0.8 ml; 40 mg/0.8 ml; and 50 mg/0.8 ml). In another embodiment, the delivery volume of methotrexate solution is advantageously held constant at about 0.7 ml, while the strength of methotrexate is varied from about 10 mg to about 50 mg (e.g., 10 mg/0.7 ml; 15 mg/0.7 ml; 20 mg/0.7 ml; 25 mg/0.7 ml; 40 mg/0.7 ml; and 50 mg/0.7 ml). In another embodiment, the delivery volume of methotrexate solution is advantageously held constant at about 0.6 ml, while the strength of methotrexate is varied from about 10 mg to about 50 mg (e.g., 10 mg/0.6 ml; 15 mg/0.6 ml; 20 mg/0.6 ml; 25 mg/0.6 ml; 40 mg/0.6 ml; and 50 mg/0.6 ml). In another embodiment, the delivery volume of methotrexate solution is advantageously held constant at about 0.5 ml, while the strength of methotrexate is varied from about 10 mg to about 50 mg (e.g., 10 mg/0.5 ml; 15 mg/0.5 ml; 20 mg/0.5 ml; 25 mg/0.5 ml; 40 mg/0.5 ml; and 50 mg/0.5 ml). In another embodiment, the delivery volume of methotrexate solution is advantageously held constant at about 0.4 ml, while the strength of methotrexate is varied from about 10 mg to about 50 mg (e.g., 10 mg/0.4 ml; 15 mg/0.4 ml; 20 mg/0.4 ml; 25 mg/0.4 ml; 40 mg/0.4 ml; and 50 mg/0.4 ml). In yet another embodiment, the delivery volume of methotrexate solution is advantageously held constant at about 0.3 ml, while the strength of methotrexate is varied from about 10 mg to about 50 mg (e.g., 10 mg/0.3 ml; 15 mg/0.3 ml; 20 mg/0.3 ml; 25 mg/0.3 ml; 40 mg/0.3 ml; and 50 mg/0.3 ml).

In a jet injector embodiment, the configuration of the jet injector and the factors affecting the injection, can be selected to obtain a Cₘₐₓ for methotrexate that is the same or substantially the same as that seen with other methods of parenteral delivery including a typical hand-powered hypodermic syringe. In another jet injector embodiment, the configuration of the injector, and the factors affecting the injection, can be selected to obtain a Tₘₐₓ for methotrexate that is the same or substantially the same as that seen with other methods of parenteral delivery, including a typical hand-powered hypodermic syringe. In a further jet injector embodiment, the configuration of the jet injector, and the factors affecting the injection, can be selected to obtain both a Cₘₐₓ and a Tₘₐₓ for methotrexate that is the same or substantially the same as that seen with other methods of parenteral delivery, including a typical hand-powered hypodermic syringe.

As would be understood by those skilled in the art, the jet injector and/or syringe as described herein may be made from any suitable materials generally used for the same. Examples of such materials include polymeric materials and glass. Non-limiting examples of polymeric materials that may be used include polypropylene, polymethylpentene, polyolefin such as cyclic polyolefin, polyethylene terephthalate, polyethylene naphthalate, noncrystalline polyarylate, PET (polyethylene terephthalate), Mitsui Plastics sold under the tradename (“TPX”), Dalkyo CZ resin and the like.

In another embodiment, methotrexate administered in accordance with the invention is delivered at a rapid rate (e.g., about 1 seconds) and controlled volume and pressure to generate a Cₘₐₓ and a Tₘₐₓ more reliably and consistently, for a dose of methotrexate ranging from about 5 mg to about 50 mg, to generate a pharmacokinetic profile, including Cₘₐₓ and Tₘₐₓ, that is substantially the same for the same dose of methotrexate when delivered via an intramuscular injection or a subcutaneous injection. Said Tₘₐₓ and Cₘₐₓ may be determined with pharmacokinetic computations and/or from blood serum or blood plasma concentration-time curves. In another embodiment, pharmacokinetic profile of methotrexate administered in accordance with the invention comprises the area under the blood serum or blood plasma concentration-time curve (AUC) of said methotrexate.

Without wishing to be bound by theory, it is believed that the constant volume of injection in combination with other variable of the needle assisted jet injection device or the powered injector or the powered jet injection device creates an approximately linear pharmacokinetic profile between increasing dose concentrations.

### V. FIGURES

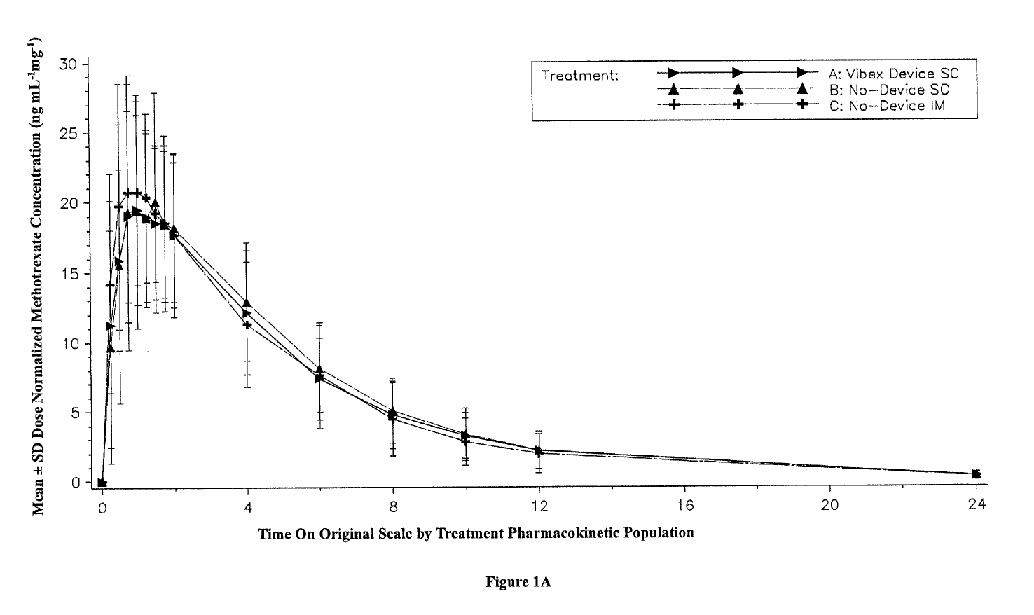

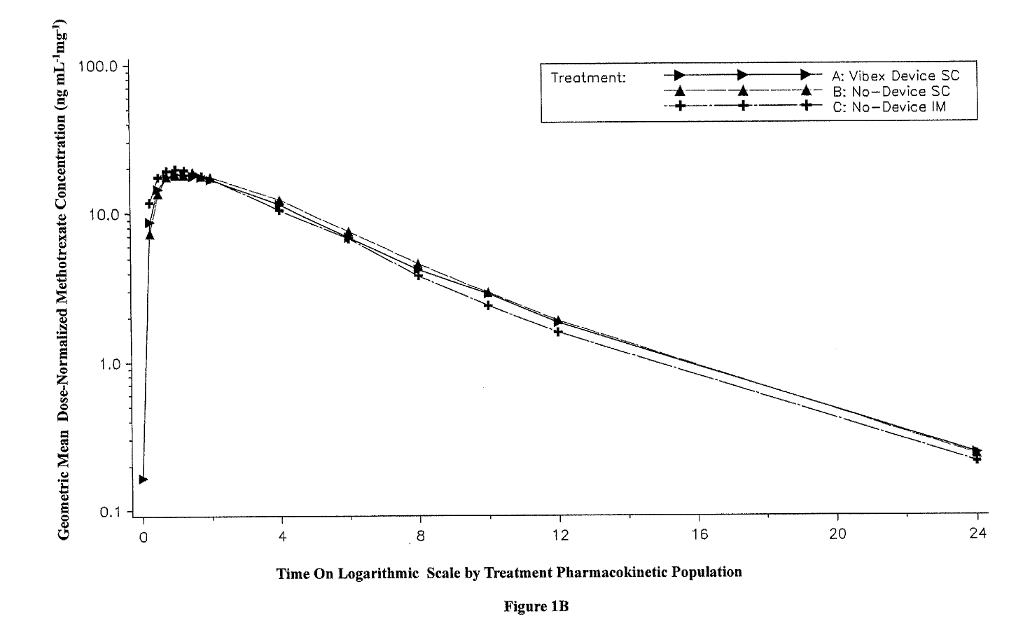

Referring to FIGS. 1A and 1B, a time course of plasma methotrexate levels when administered subcutaneously with the Vibex™ device versu_{s w}hen adminᵢₛₜered subcutaneously or intramuscularly by a needle and a syringe is shown. FIG. 1A shows a time course of plasma methotrexate levels when administered with the Vibex™ subcutaneously versus when administered by a needle and a syringe subcutaneously or intramuscularly. FIG. 1A is a plot of mean dose-normalized plasma methotrexate concentration versus time on original scale by treatment. FIG. 1B shows a plot of geometric mean dose-normalized plasma methotrexate concentration versus time on a logarithmic scale by treatment. Both FIGS. 1A and 1B show that methotrexate delivered subcutaneously by a needle assisted jet injection device (Vibex™ SC) has substantially the same pharmacokinetic profile as the same methotrexate dose administered subcutaneously or intramuscularly with the aid of a needle and syringe (respectively referred to as No-Device IM and No-Device SC).

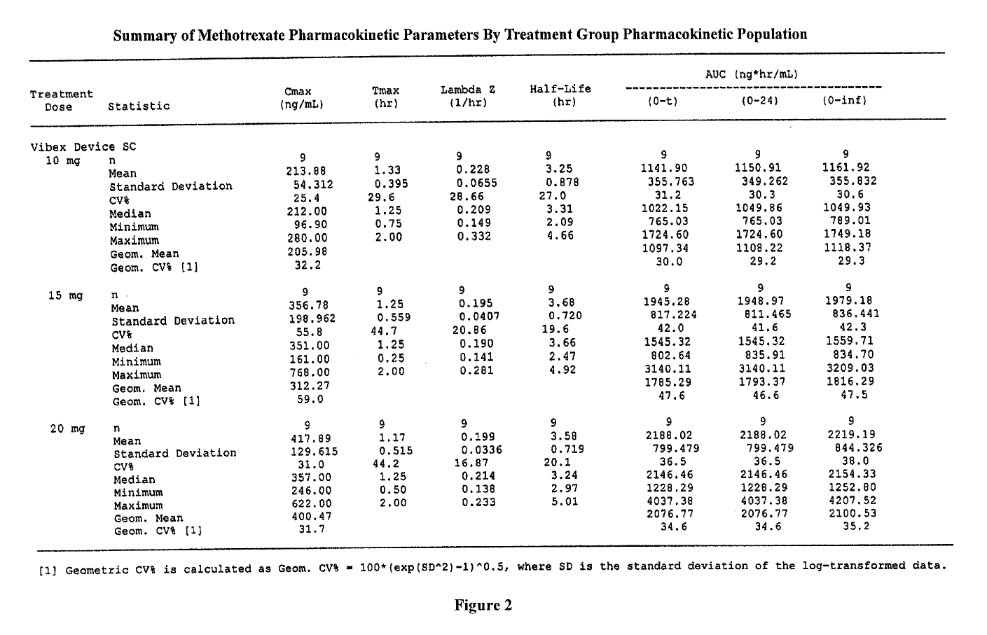

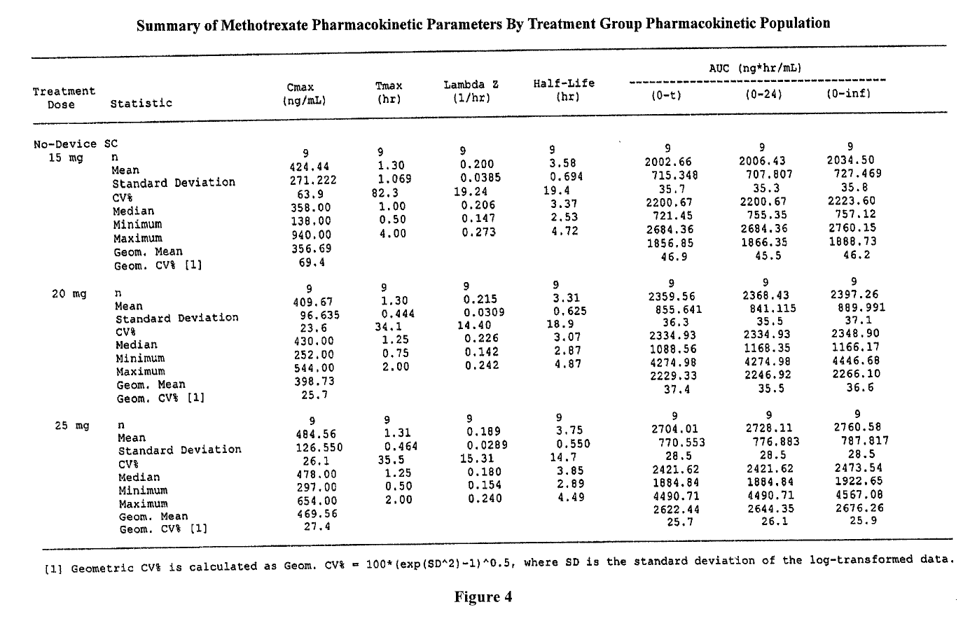

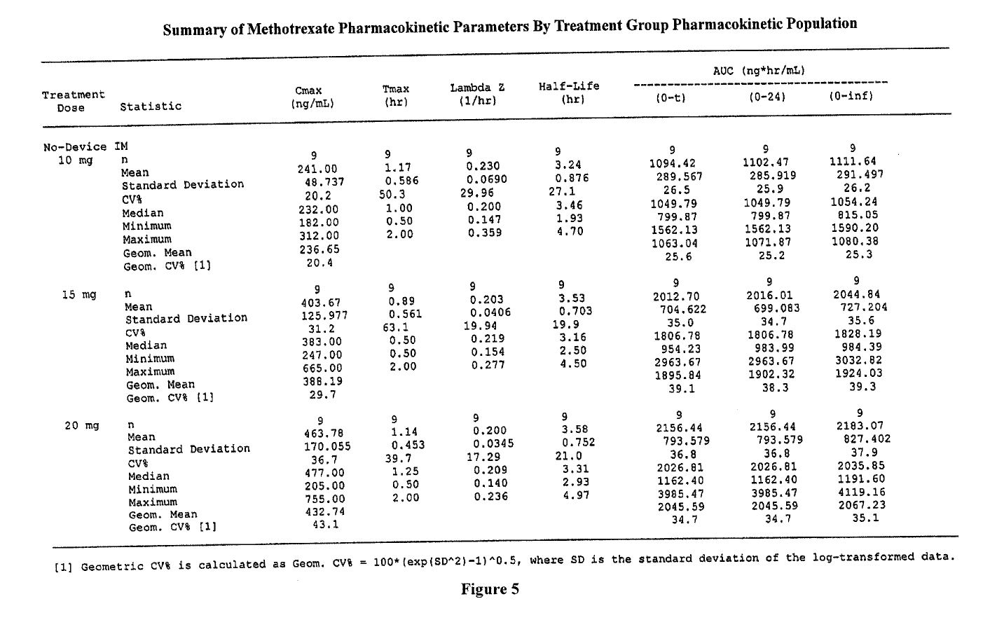

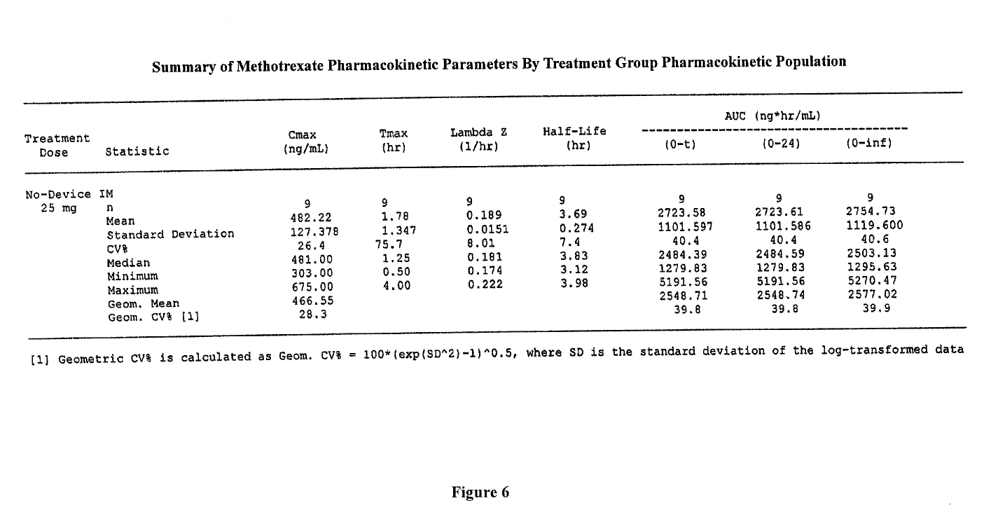

As shown in FIGS. 1A and 1B, pharmacokinetic parameters, including Tₘₐₓ, AUC and Cₘₐₓ, estimated based on the observed pharmacokinetic profiles of methotrexate provided the various treatment methods also appear to be substantially the same or similar. FIGS. 2-6 provide pharmacokinetic data for subjects with rheumatoid arthritis that were subcutaneously and intramuscularly administered a single dose of methotrexate in dosage strengths of about 10 mg, about 15 mg, about 20 mg and about 25 mg. The pharmacokinetic data include values for the observed maximum plasma concentration (Cₘₐₓ), total area under the plasma concentration-time curve (AUC₍₀₋ₜ₎, AUC₍₀₋₂₄₎, AUC_{(0-inf)}) and the time to ₘₐₓimum plasma concentration (Tₘₐₓ).

Referring to FIG. 2, a summary of pharmacokinetic parameters of methotrexate, observed following subcutaneous administration of a dosage of 10 mg, 15 mg, and 20 mg utilizing the Vibex™ device.

By way of example, methotrexate was advantageously administered subcutaneously with Vibex™ device in a dosage of 10 mg to provide mean Cₘₐₓ of about 213 ng/ml, AUC(0-t) of about 1141 ng*hr/ml, AUC(0-24) of about 1150 ng*hr/ml, AUC(O-inf) of about 1161 ng*hr/ml, Tₘₐₓ 1.33 hours, and a half-life of about 3 hours.

By way of example, methotrexate was advantageously administered subcutaneously with a Vibex™ device, in a dosage of 10 mg to provide mean Cₘₐₓ of from about 170 ng/ml to about 266 ng/ml, AUC₍₀₋ₜ₎ of from about 912 ng*hr/ml to about 1426 ng*hr/ml, AUC₍₀₋₂₄₎ of from about 920 ng*hr/ml to about 1437 ng*hr/ml, AUC_{(0-inf)} is from about 929 ng*hr/ml to about 1451 ng*hr/ml, Tₘₐₓ of from about 1.06 hours to about 1.66 hours, and a half-life of from about 2.6 hours to about 4.06 hours.

By way of example, methotrexate was advantageously administered subcutaneously with a Vibex™ device, in a dosage of 15 mg to provide a mean Cₘₐₓ of about 356 ng/ml, an AUC₍₀₋ₜ₎ of about 1945 ng*hr/ml, an AUC₍₀₋₂₄₎ of about 1948 ng*hr/ml, an AUC_{(0-inf)} of about 1979 ng*hr/ml, a Tₘₐₓ of about 1.25 hours, and a half-life of about 3.68 hours.

By way of example, methotrexate was advantageously administered subcutaneously with a Vibex™ device in a dosage of 15 mg to provide a mean Cₘₐₓ of from about 284 ng/ml to about 445 ng/ml, an AUC₍₀₋ₜ₎ of from about 1556 ng*hr/ml to about 2435 ng*hr/ml, an AUC₍₀₋₂₄₎ of from about 1558 ng*hr/ml to about 2435 ng*hr/ml, an AUC_{(0-inf)} of from about 1583 ng*hr/ml to about 2473 ng*hr/ml, a Tₘₐₓ of from about 1 hour to about 1.56 hours, and a half-life of from about 2.94 hours to about 4.60 hours.

By way of example, methotrexate was advantageously administered subcutaneously with a Vibex™ device, in a dosage of 20 mg to provide a mean Cₘₐₓ of about 417 ng/ml, an AUC₍₀₋ₜ₎ of about 2188 ng*hr/ml, an AUC₍₀₋₂₄₎ of about 2188 ng*hr/ml, an AUC_{(0-inf)} of about 2219 ng*hr/ml, a Tₘₐₓ of about 1.17 hours, and a half-life of about 3.58 hours.

By way of example, methotrexate was advantageously administered subcutaneously with a Vibex™ device, in a dosage of 20 mg to provide a mean Cₘₐₓ of from about 333 ng/ml to about 521 ng/ml, an AUC₍₀₋ₜ₎ of from about 1750 ng*hr/ml to about 2735 ng*hr/ml, an AUC₍₀₋₂₄₎ of from about 1750 ng*hr/ml to about 2735 ng*hr/ml, an AUC_{(0-inf)} of from about 1775 ng*hr/ml to about 2773 ng*hr/ml, a Tₘₐₓ of from about 0.93 hours to about 1.46 hours, and a half-life of from about 2.86 hours to about 4.47 hours.

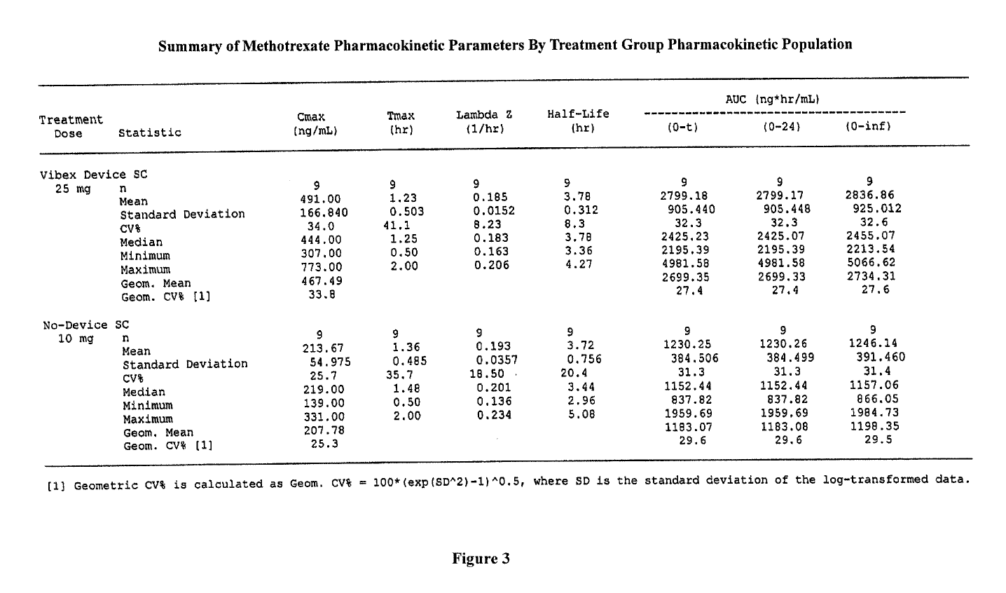

Referring to FIG. 3, a summary of pharmacokinetic parameters of methotrexate, observed following subcutaneous administration utilizing a Vibex™ device, in a dosage of 25 mg to provide a mean Cₘₐₓ of about 491 ng/ml, an AUC₍₀₋ₜ₎ of about 2799 ng*hr/ml, an AUC₍₀₋₂₄₎ of about 2799 ng*hr/ml, an AUC_{(0-inf)} of about 2836 ng*hr/ml, a Tₘₐₓ of about 1.23 hours, and a half-life of about 3.78 hours.

By way of example, methotrexate was advantageously administered subcutaneously with a Vibex™ device, in a dosage of 25 mg to provide a mean Cₘₐₓ of from about 392 ng/ml to about 613 ng/ml, an AUC₍₀₋ₜ₎ of from about 2239 ng*hr/ml to about 3498 ng*hr/ml, an AUC₍₀₋₂₄₎ of from about 2239 ng*hr/ml to about 3498 ng*hr/ml, an AUC_{(0-inf)} of from about 2268 ng*hr/ml to about 3545 ng*hr/ml, a Tₘₐₓ of from about 0.98 hours to about 1.54 hours, and a half-life of from about 3.02 hours to about 4.72 hours.

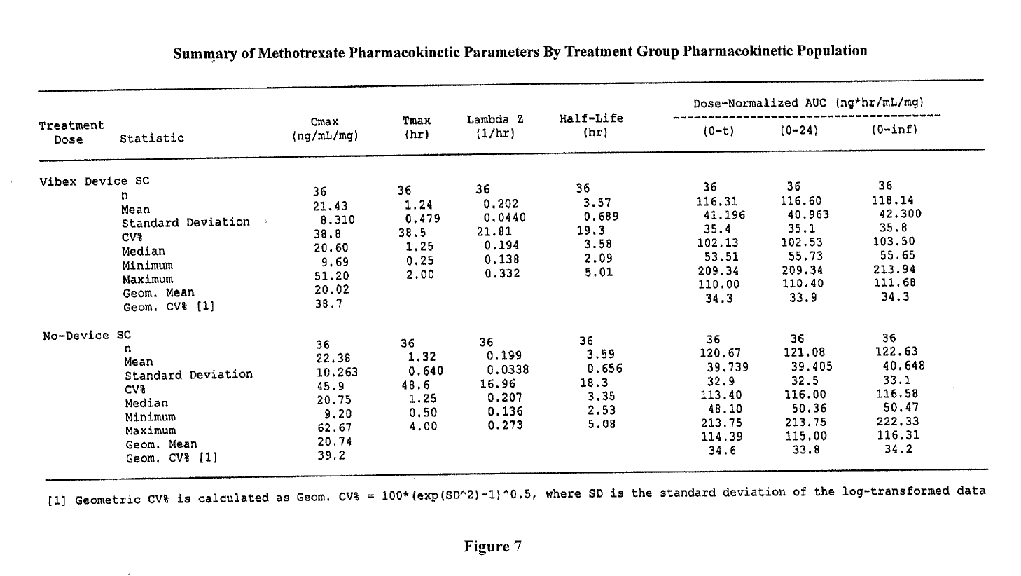

Referring to FIG. 7, a summary of dose-normalized pharmacokinetic parameters methotrexate by Treatment Group (Vibex™ device SC treatment group and needle and syringe SC group) is shown. FIG. 7 demonstrates that the dose-normalized pharmacokinetic profiles of methotrexate administered subcutaneously by the Vibex™ device and by needle and syringe are substantially similar. In particular, the observed pharmacokinetic parameters for both treatments, including dose-normalized Cₘₐₓ (ng/ml/mg), Tₘₐₓ (hr), half life (hr), and dose-normalized AUC₍₀₋ₜ₎ (ng*hr/ml/mg), AUC₍₀₋₂₄₎ (ng*hr/ml/mg), and AUC_{(0-inf)} (ng*hr/ml/mg).

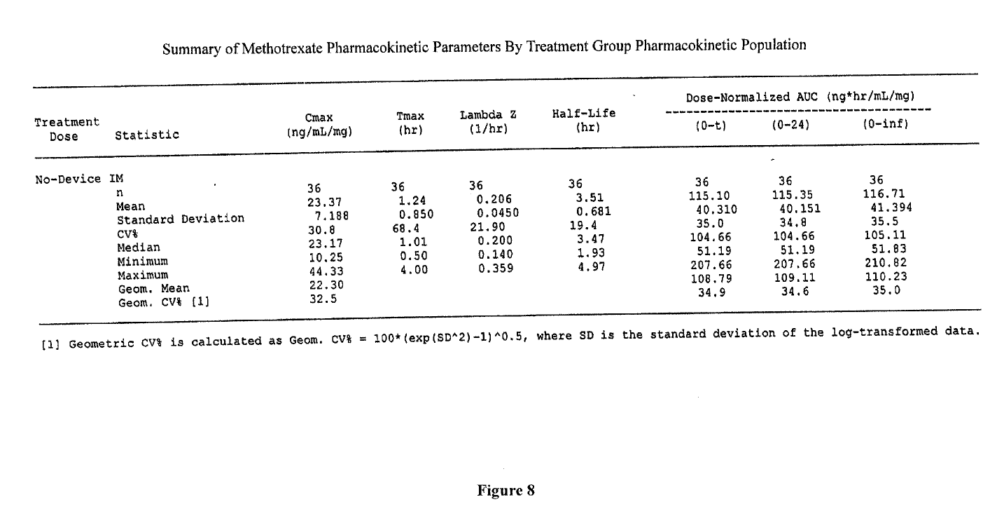

Referring to FIG. 8, a summary of dose-normalized pharmacokinetic parameters of methotrexate by Treatment Group, observed following intramuscular administration of methotrexate utilizing syringe and needle is shown. FIG. 7 also demonstrates that the dose-normalized pharmacokinetic profile of methotrexate administered intramuscularly by the needle and syringe is substantially the same or similar to the pharmacokinetic profiles observed when methotrexate is administered subcutaneously either by the Vibex™ device or by needle and syringe. In particular, the observed dose-normalized pharmacokinetic parameters for intramuscular administration of methotrexate, including dose-normalized Cₘₐₓ (ng/ml/mg), Tₘₐₓ (hr), half life (hr), and dose-normalized AUC₍₀₋ₜ₎ (ng*hr/ml/mg), AUC₍₀₋₂₄₎ (ng*hr/ml/mg), and AUC_{(0-inf)} (ng*hr/ml/mg) are substantially similar to the corresponding pharmacokinetic parameters observed when methotrexate was administered subcutaneously by the Vibex™ Device or by syringe and needle.

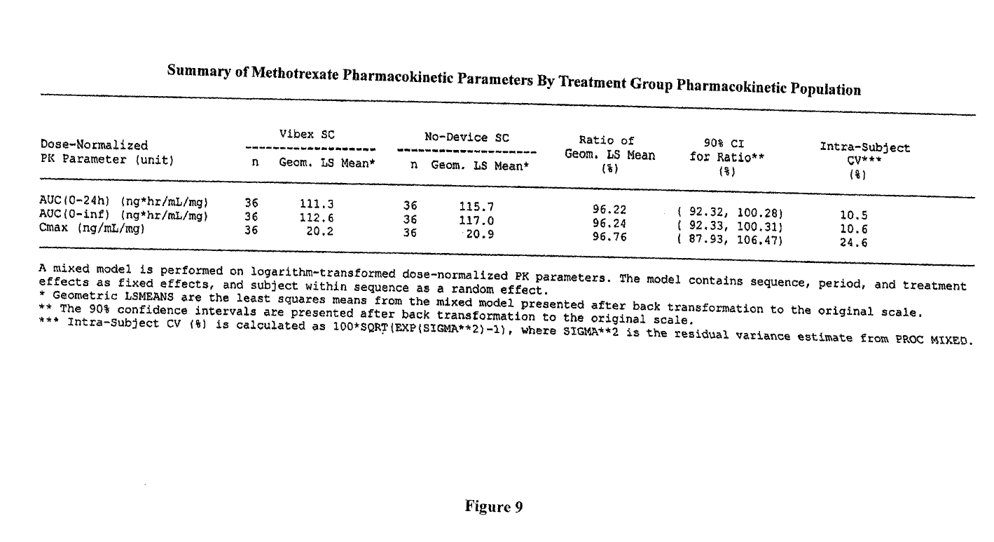

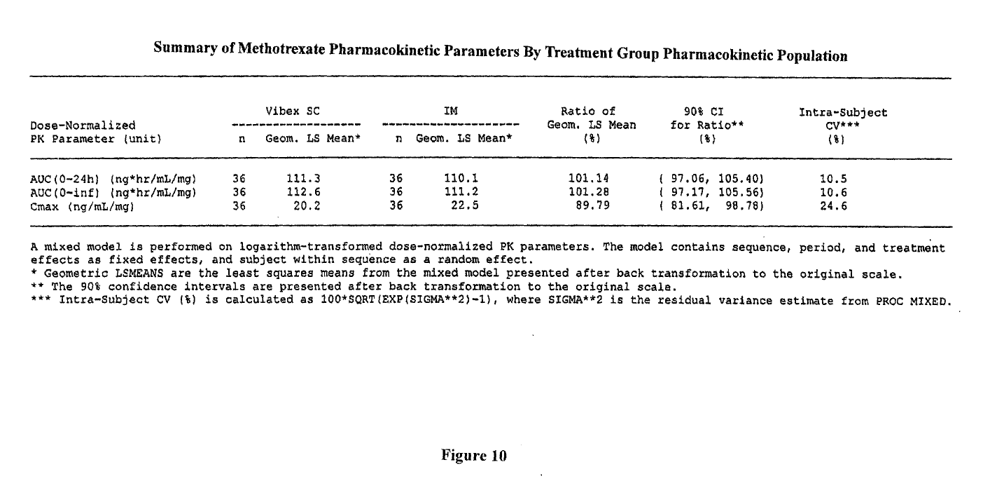

FIGS. 9 and 10 provide a summary of mixed-model analysis of dose-normalized methotrexate pharmacokinetic parameters by treatment group, namely with a Vibex™ device subcutaneously, or with needle and syringe, subcutaneously or intramuscularly.

FIG. 9 shows comparative analysis of methotrexate administration via Vibex™ Device, versus subcutaneous injection with needle and syringe. Bioequivalence ratios of the geometric LS means of the AUC₍₀₋₂₄₎/Dose, AUC_{(0-inf)}/Dose, and Cₘₐₓ/Dose PK parameters for Vibex™ Device, and subcutaneous injection with needle and syringe were:
AUC₍₀₋₂₄₎/Dose geometric LS mean ratio was 96% with a 90% CI of (92.3%, 100.3%);
AUC_{(0-inf)}/Dose geometric LS mean ratio was 96% with a 90% CI of (92.3%, 100.3%); and
Cₘₐₓ/Dose geometric LS mean ratio was 97% with a 90% CI of (87.9%, 106.5%).
   Since the confidence intervals (CIs) of all ratios were contained within the bioequivalence range of 80% to 125%, Vibex™ Device was bioequivalent to subcutaneous injection with needle and syringe.

FIG. 10 shows comparative analysis of methotrexate administration via Vibex™ Device, versus intramuscular injection with needle and syringe. Bioequivalence ratios of the geometric LS means of the AUC₍₀₋₂₄₎/Dose, AUC_{(0-inf)}/Dose, and Cₘₐₓ/Dose PK parameters for Vibex™ Device, and intramuscular injection with needle and syringe were:
AUC₍₀₋₂₄₎/Dose geometric LS mean ratio was 101% with a 90% CI of (97.1%, 105.4%);
AUC_{(0-inf)}/Dose geometric LS mean ratio was 101% with a 90% CI of (97.2%, 105.6%); and
Cₘₐₓ/Dose geometric LS mean ratio was 90% with a 90% CI of (81.6%, 98.8%).
   Since the CIs of all ratios were contained within the bioequivalence range of 80% to 125%, Vibex™ Device was bioequivalent to intramuscular injection with needle and syringe.

These findings are unexpected since previous studies such as Medac GmbH in support of its request for marking Metoject® 50 mg/ml in Sweden, “[t]he administration of the higher strength (50 mg/ml) of MTX [methotrexate] resulted in similar total exposure in terms of AUC, but somewhat higher Cₘₐₓ (15-20% higher), compared with the marketed lower strength (10 mg/ml) following both i.m. and s.c. administration.

Referring to FIGS. 2-6, the pharmacokinetics of methotrexate delivered according to the methods of the invention have been found to be substantially similar to the pharmacokinetics of conventional SC delivery of the drug, indicating that Vibex™ SC administration according to the methods of the invention will provide improved clinical results. Also, the observed plasma levels of methotrexate appeared to increase linearly in a dose-proportionate manner after single-dose administration of the methotrexate within the 10 mg to 25 mg dose range. These findings are pertinent not only to methotrexate in the 10 mg to 25 mg dose range, but also to methotrexate in the 5 mg to 50 mg dose range and/or in the 7.5 mg to 50 mg dose range. Accordingly, in an aspect, the present invention provides a method of treating an autoimmune disorder in a subject in need of treatment, said method comprising introducing into the subcutaneous tissue of said subject, from a needle assisted jet injection device, a composition comprising methotrexate in dose ranging from about 5 mg to 50 mg dose range or 7.5 mg to about 50 mg, wherein said method provides plasma levels of methotrexate in the blood plasma of the subject that increase linearly in a dose-proportionate manner.

The invention encompasses methods of delivering methotrexate to the subcutaneous compartment so that the amount of the pre-selected dose of methotrexate deposited in the subcutaneous tissue is increased compared to when methotrexate is administered via a hand-powered syringe, for example. Directly targeting the subcutaneous compartment as taught by the invention provides more rapid onset of effects of methotrexate and comparable or higher bioavailability including tissue bioavailability, relative to other modes of delivery of methotrexate. In addition, the invention suggests comparable or better responses to regular needle delivery for the subcutaneous methotrexate application, the improvement in methotrexate bioavailability may be due to more reliable and consistent delivery of methotrexate that provides consistent pharmacokinetic profile of the methotrexate. Methotrexate delivered in accordance with the methods of the invention can be rapidly absorbed and systemically distributed via controlled subcutaneous injection that selectively accesses the subcutaneous vascular system, thus methotrexate may exert its beneficial effects more rapidly than oral administration, for example.

These and other aspects of the present invention will be further appreciated upon consideration of the following Examples, which are intended to illustrate certain particular embodiments of the invention but are not intended to limit its scope, as defined by the claims.

### VI. BIOAVAILABILITY TESTING

Isotonicity of the Vibex™ Device product was adjusted over the 10 mg through to 25 mg doses by varying the sodium chloride level to compensate for the increase in the drug concentration with increase methotrexate dose since the dose volume was constant. Among the parenteral formulation factors that should be of greatest concern is osmolarity. Generally, parenteral products should be formulated to as close a physiological osmolarity as possible. Consequences of injections with non-physiological osmolarity include injection pain, inflammatory processes at, the site of injection (e.g., phlebitis and cellulitis), and hemolysis when injecting hypotonic products. Further consequences of these inflammatory processes include vein damage, extravasation, emboli formation following phlebitis, and tissue necrosis and gangrene following cellulitis. Therefore, it is important to control the osmolarity.

pH of the Vibex™ Device product was adjusted with sodium hydroxide to be 8-8.5. It is important to have the pH close to physiological pH (7.4) to prevent stinging, burning, pain, irritation, or tissue damage.

The following formulations containing methotrexate were prepared to compare bioavailability of methotrexate utilizing various injection methods (subcutaneous with a syringe, intramuscular with a syringe, and subcutaneous with needle assisted jet injector).

A. Injectable Solutions of Methotrexate

Injectable solution (10 mg) containing methotrexate; sodium chloride (1.96 mg); sodium hydroxide (adjustment to pH 8.2-8.5); and water (q.s. ad 0.4 mL). The unit dose injectable solution, containing methotrexate disodium corresponds to 10 mg methotrexate per 0.4 ml. Based on certificate of analysis, the actual content of the injectable solution was assayed at 9.98 mg (99.8% of 10 mg).

Injectable solution (15 mg) containing methotrexate; sodium chloride (1.6 mg); sodium hydroxide (adjustment to pH 8.2-8.5); and water (q.s. ad 0.4 ml). The unit dose injectable solution, containing methotrexate disodium corresponds to 15 mg methotrexate per 0.4 ml. Based on certificate of analysis, the actual content of the injectable solution was assayed at 15.47 mg (103.1% of 15 mg).

Injectable solution (20 mg) containing methotrexate; sodium chloride (1.28 mg); sodium hydroxide (adjustment to pH 8.2-8.5); and water (q.s. ad 0.4 ml). The unit dose injectable solution, containing methotrexate disodium corresponds to 20 mg methotrexate per 0.4 ml. Based on certificate of analysis, the actual content of the injectable solution was assayed at 20.20 mg (101.1% of 20 mg).

Injectable solution (25 mg) containing methotrexate; sodium chloride (0.56 mg); sodium hydroxide (adjustment to pH 8.2-8.5); and water (q.s. ad 0.4 ml). The unit dose injectable solution, containing methotrexate disodium corresponds to 25 mg methotrexate per 0.4 ml. Based on certificate of analysis, the actual content of the injectable solution was assayed at 25.88 mg (103.5% of 25 mg).

The bioavailability was evaluated during a three period, crossover study of the relative bioavailability of methotrexate injectable solutions injected using a needle assisted jet injection device (Vibex™ device SC) versus injection with a syringe (no device SC) and intramuscular injection (No Device IM).

This was a randomized, open-label, 3-way crossover study involving 36 subjects. During the screening period, 1 of 4 MTX dose groups (10 mg, 15 mg, 20 mg, or 25 mg) were selected by the investigator for each subject based on the subject's current MTX dose at the time of enrollment and disease status (controlled RA vs. uncontrolled RA). Subjects received a single dose of methotrexate on three separate occasions (Day 1 of each of three periods), with a minimum 7-day washout, which started from the day of dosing of each period. Subjects received four dosage levels of methotrexate (10 mg, 15 mg dose, 20 mg and 25 mg) by three different methods of administration (Vibex™ device SC, No-device SC and No-Device IM) during each of the three periods. Each period consisted of a single dosing of methotrexate and 1 day of study assessments. The order in which the subjects received each single dose of methotrexate was determined by the treatment sequence to which the subject was randomized. One of six treatment sequences was possible as determined by the randomization schedule.

| Sequence | Period 1 | Period 2 | Period 3 |
|---|---|---|---|
| 1 | A | B | C |
| 2 | A | C | B |
| 3 | B | C | A |
| 4 | B | A | C |
| 5 | C | A | B |
| 6 | C | B | A |

The randomization sequence for the enrolled subjects is as follows.

| | | | Treatment |
|---|---|---|---|
| | Patient | Dose Group | Randomization |
| PI-Site# | Number | (mg of MTX) | Sequence* |
| 001 | 001 | 10 | BAC |
| 001 | 002 | 10 | ABC |
| 001 | 009 | 10 | CAB |
| 001 | 011 | 10 | ACB |
| 001 | 012 | 10 | CBA |
| 001 | 023 | 10 | BCA |
| 001 | 024 | 10 | BCA |
| 001 | 028 | 10 | BAC |
| 001 | 045 | 10 | ACB |
| 001 | 007 | 15 | CBA |
| 001 | 015 | 15 | CAB |
| 001 | 016 | 15 | ABC |
| 001 | 017 | 15 | BCA |
| 001 | 026 | 15 | BCA |
| 001 | 033 | 15 | CBA |
| 001 | 037 | 15 | ABC |
| 002 | 001 | 15 | ACB |
| 002 | 002 | 15 | BAC |
| 001 | 005 | 20 | ABC |
| 001 | 008 | 20 | CAB |
| 001 | 018 | 20 | ACB |
| 001 | 020 | 20 | BCA |
| 001 | 025 | 20 | BAC |
| 001 | 027 | 20 | CBA |
| 001 | 029 | 20 | ABC |
| 001 | 031 | 20 | BCA |
| 001 | 044 | 20 | CAB |
| 001 | 003 | 25 | CBA |
| 001 | 032 | 25 | ACB |
| 001 | 035 | 25 | BCA |
| 001 | 036 | 25 | ABC |
| 001 | 039 | 25 | CAB |
| 001 | 040 | 25 | BCA |
| 001 | 041 | 25 | BAC |
| 001 | 043 | 25 | CAB |
| 001 | 046 | 25 | BAC |

*A = Vibex ™ Device; B = subcutaneous needle and syringe; C = intramuscular needle and syringe.

Blood samples for pharmacokinetic analysis of methotrexate were collected prior to each administration of study medication and at specified time points during the 24-hour period following study medication administration, depending on whether the method of administration is Vibex™ device SC, No-device SC or No-Device IM. A total of approximately 200 ml blood was obtained from each subject over the course of the study. For each period, subjects were confined to the clinical facility for day 1 for 12-hours and returned for 24-hour postdose sample collection, and blood samples were collected as follows: predose and 0.25, 0.5, 0.75, 1.0, 1.25, 1.5, 1.75, 2.0, 4.0, 6.0, 8.0, 10.0, 12.0 and 24 hour postdose administration.

B. Sample Collection

Blood samples (4 ml) were drawn into a tube containing sodium heparin at the following times relative to dosing with Methotrexate as follows: predose and 0.25, 0.5, 0.75, 1.0, 1.25, 1.5, 1.75, 2.0, 4.0, 6.0, 8.0, 10.0, 12.0 and 24 hour postdose administration. The same schedule was followed for all tested methods for administration of methotrexate. Immediately following collection of each sample, each tube was gently inverted and placed in ice. Within 30 minutes of sample collection, the tubes were centrifuged at about 2000×G for 15 minutes at approximately 4° C. in order to separate the cells from the plasma. No aids for separation were used. The plasma was transferred with clean pipettes and placed in two polypropylene storage tubes in equal volumes. The storage tubes were labeled with the following information: protocol number, subject number, period number, relative time of sample, and analyte. The label was taped with clear tape to assure adherence of the label. The storage tubes were placed in a freezer at 70° C. or below until they analyzed.

C. Bioanalytical Methodology

Plasma samples were assayed for methotrexate concentrations using a validated and sensitive LC/MS/MS (liquid chromatography/mass spectrometry/mass spectrometry) method. Briefly, in an ice water bath, 100 μL of the human K₂EDTA plasma sample was placed in a 2-ml centrifuge tube. 10 μL of working standard solution and 10 μL of internal standard were added to the tubes. Afterwards, 10 μA of 30% formic acid in water was added to the tubes and 0.2 ml of cold precipitation solution (acetonitrile) was added, vortexed and centrifuged. The supernatant was transferred to a 96-well plate for LC/MS/MS analysis. A volume of approximately 5 μL of the sample was injected into an LC/MS/MS system. The LC/MS/MS analysis was carried out with a Sciex API-4000 mass spectrometer coupled with a Shimadzu LC system. Concentrations were calculated using an eight-point curve ranging from 1 to 1000 ng/ml for methotrexate with weighted linear regression. Calibration curves and quality control samples were included in each run. The ranges of the assay for both analyses were from 1 ng/ml to 1000 ng/ml, and the limit of quantification for both analyses was 1 ng/ml. Calibration standards used in the study batches were prepared fresh in human plasma on the day of analysis at nominal concentrations of 1, 2, 10, 25, 100, 250, 500, and 1000 ng/ml of methotrexate. Methotrexate QC samples at low, mid, and high concentrations (3, 40, and 900 ng/ml, respectively) were prepared freshly and in a batch and stored at nominal −70° C. The intra assay precision, expressed as a coefficient of variation (% CV), for the lower quality control standard was 8.3% for methotrexate. The inter-day CV and mean bias values for the methotrexate calibration standards in human plasma ranged from 4.7% to 7.3% and from −3.9% to 3.0%, respectively. The coefficients of determination (r²) for these analytical batches were ≧0.9908 for methotrexate in human plasma.

Concentration values that were reported as below the limit of quantification (BLQ) were treated as 0 for computing mean concentrations and in the concentration-time graphs. For pharmacokinetic analyses of methotrexate, BLQ results were included in the input file. Concentrations in plasma were used as reported; but measurable concentrations observed after obtaining a BLQ result were excluded from analysis.

D. Pharmacokinetic Measurements

Methotrexate concentrations in plasma were entered into a spreadsheet.

The Linear Up Log Down method (equivalent to the Linear Up/Log Down option in WinNonlin® Professional) was used in the computation of AUCs. The linear trapezoidal method was employed for any area where the concentration data was increasing (or constant) and the logarithmic trapezoidal method for any area where the concentration data was decreasing. Interpolation of concentration values was conducted using the linear interpolation rule for any time point surrounded by points of increasing (or constant) concentrations and the logarithmic interpolation rule for any time point surrounded by points of decreasing concentrations.

Pharmacokinetic parameters were calculated from the individual concentrations of MTX using noncompartmental methods. The primary PK parameters calculated for each treatment included the following: dose-normalized area under the curve from time zero to infinity (AUC_{(0-inf)}/Dose), dose-normalized area under the curve from time zero to the last measurable concentration (AUC₍₀₋ₜ₎/Dose) and dose-normalized maximum observed concentration (Cₘₐₓ/Dose). Since the study was designed to provide evidence for the comparability of the Vibex^{MTX} device to SC injection without device and IM injection over a dose range of 10 mg to 25 mg, the maximum observed concentration (Cₘₐₓ) and area under the curve (AUC) results for each treatment were dose-normalized to allow data from all dose groups (10 mg, 15 mg, 20 mg, and 25 mg) in a given treatment (Treatment A, Treatment B, or Treatment C) to be pooled. Secondary PK parameters calculated for each treatment were the following: time of maximum concentration (t . . . ), half-life (tin), Lambda z, and percent of area under the curve extrapolated.

Comparisons among treatments were evaluated by a mixed model analysis of the log-transformed, dose-normalized values of Cₘₐₓ, AUC₍₀₋ₜ₎, and AUC_{(0-inf)} with sequence, treatment, and treatment period as fixed effects and subject nested within sequence as a random effect. Using these models, the least-squares (LS) mean for the above PK parameters of each treatment were determined. The differences in LS means among treatments and the 90% confidence intervals (CIs) for the differences in the log-scale LS means among treatment were also obtained. The results were transformed back to the original scale by exponentiation to provide geometric LS means for each treatment.

The following ratios of the geometric LS means for Cₘₐₓ/Dose, area under the curve from time zero to 24 hours (AUC₍₀₋₂₄₎/Dose), and AUC_{(0-inf)}/Dose were performed to determine relative bioavailability:
Vibex™ Device geometric LS mean/subcutaneous with needle and syringe geometric LS mean and
Vibex™ Device geometric LS mean/intramuscular injection with needle and syringe geometric LS mean.

Vibex™ Device was considered bioequivalent to subcutaneous needle and syringe and intramuscular needle and syringe if the 90% CI for the ratio of the Cₘₐₓ/Dose geometric LS means fell within 80% to 125% and the 90% CI for the ratio of the AUC_{(0-inf)}/Dose geometric LS means fell within 80% to 125%. These criteria were achieved as described below. FDA guidelines for bioequivalence were met. No significant differences between Vibex™ Device SC/No-Device SC, and Vibex™ Device SC/No-Device IM were demonstrated. The results of this study demonstrate that low dose subcutaneous administration of methotrexate using the needle assisted jet injection device of the invention is sufficient to provide pharmacokinetic profile of methotrexate that is and not significantly different from that of the same dose of methotrexate when delivered using an intramuscular injection or a subcutaneous injection.

The pharmacokinetic parameters obtained for methotrexate from the Linear Up/Log Down option in WinNonlin® Professional are shown in FIGS. 1-8.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

It should be understood that various changes, substitutions, and alterations can be made herein without departing from the spirit and scope of the invention as defined by the appended claims. Moreover, the scope of the present application is not intended to be limited to the particular embodiments of the process, machine, manufacture, and composition of matter, means, methods and steps described in the specification. As one of ordinary skill in the art will readily appreciate from the disclosure herein, processes, machines, manufacture, composition of matter, means, methods, or steps, presently existing or later to be developed that perform substantially the same function or achieve substantially the same result as the corresponding embodiments described herein may be utilized according to the present invention.

1. A method of treating an autoimmune disorder in a subject in need of treatment with a composition comprising methotrexate, said method comprising:
introducing into the subcutaneous tissue of said subject, from a needle assisted jet injection device, a composition comprising methotrexate in a dose ranging from about 5 mg to about 50 mg,
wherein the pharmacokinetic profile of said methotrexate, obtained following administration of said methotrexate by said needle assisted jet injection device, is substantially the same as the pharmacokinetic profile of the same dose of said methotrexate when administered by an intramuscular injection or a subcutaneous injection.

2. The method of claim 1, wherein said pharmacokinetic profile comprises a set of one or more pharmacokinetic parameters selected from the group consisting of:
(a) bioavailability of said methotrexate following said administration by said needle assisted jet injection device;
(b) time of peak concentration (Tₘₐₓ) of a blood (serum or plasma) concentration-time curve of said methotrexate following said administration by said needle assisted jet injection device;
(c) peak height concentration (Cₘₐₓ) of a blood (or serum or plasma) concentration time curve of said methotrexate following said administration by said needle assisted jet injection device;
(d) area under a blood (serum or plasma) concentration-time curve (AUC) of said methotrexate following administration by said needle assisted jet injection device; and
(e) half-life of said methotrexate following administration by said needle assisted jet injection device;
(f) combinations of (a), (b), (c), (d) and (e)

3. The method of claim 2, wherein said Cₘₐₓ has a value selected from the group consisting of: from about 170 ng/ml to about 266 ng/ml when the dose of methotrexate is about 10 mg; from about 284 ng/ml to about 445 ng/ml when the dose of methotrexate is about 15 mg; from about 333 ng/ml to about 521 ng/ml when the dose of methotrexate is about 20 mg; and from about 392 ng/ml to about 613 ng/ml when the dose of methotrexate is about 25 mg.

4. The method of claim 3, wherein said Cₘₐₓ has a value selected from the group consisting of: about 213 ng/ml when the dose of methotrexate is about 10 mg; about 356 ng/ml when the dose of methotrexate is about 15 mg; about 417 ng/ml when the dose of methotrexate is about 20 mg; and about 491 ng/ml when the dose of methotrexate is about 25 mg.

5. The method of claim 2, wherein said AUC is selected from the group consisting of: AUC₍₀₋ₜ₎ of from about 912 ng*hr/ml to about 1426 ng*hr/ml when the dose of methotrexate is about 10 mg; an AUC₍₀₋₂₄₎ of from about 920 ng*hr/ml to about 1437 ng*hr/ml when the dose of methotrexate is about 10 mg; and an AUC_{(0-inf)} is from about 929 ng*hr/ml to about 1451 ng*hr/ml when the dose of methotrexate is about 10 mg; and combinations thereof.

6. The method of claim 2, wherein said AUC is selected from the group consisting of AUC₍₀₋ₜ₎ of from about 1556 ng*hr/ml to about 2435 ng*hr/ml when the dose of methotrexate is about 15 mg; an AUC₍₀₋₂₄₎ of from about 1558 ng*hr/ml to about 2435 ng*hr/ml when the dose of methotrexate is about 15 mg; an AUC_{(0-inf)} of from about 1583 ng*hr/ml to about 2473 ng*hr/ml when the dose of methotrexate is about 15 mg; and combinations thereof.

7. The method of claim 2, wherein said AUC is selected from the group consisting of: an AUC₍₀₋ₜ₎ of from about 1750 ng*hr/ml to about 2735 ng*hr/ml when the dose of methotrexate is about mg; an AUC₍₀₋₂₄₎ of from about 1750 ng*hr/ml to about 2735 ng*hr/ml when the dose of methotrexate is about 20 mg; an AUC_{(0-inf)} of from about 1775 ng*hr/ml to about 2773 ng*hr/ml when the dose of methotrexate is about 20 mg; and combinations thereof.

8. The method of claim 2, wherein said AUC is selected from the group consisting of: an AUC₍₀₋ₜ₎ of from about 2239 ng*hr/ml to about 3498 ng*hr/ml when the dose of methotrexate is about mg; an AUC₍₀₋₂₄₎ of from about 2239 ng*hr/ml to about 3498 ng*hr/ml when the dose of methotrexate is about 25 mg; and an AUC_{(0-inf)} of from about 2268 ng*hr/ml to about 3545 ng*hr/ml when the dose of methotrexate is about 25 mg.

9. The method of claim 2, wherein said set of one or more pharmacokinetic parameters is selected from the group consisting of: a Tₘₐₓ of from about 1.06 hours to about 1.66 hours when the dose of methotrexate is about 10 mg; a half-life of from about 2.6 hours to about 4.06 hours when the dose of methotrexate is about 10 mg; and a combination thereof.

10. The method of claim 2, wherein said set of one or more pharmacokinetic is selected from the group consisting of: a Tₘₐₓ of from about 1 hour to about 1.56 hours is when the dose of methotrexate is about 15 mg; a half-life of from about 2.94 hours to about 4.60 hours when the dose of methotrexate is about 15 mg; and a combination thereof.

11. The method of claim 2, wherein said set of one or more pharmacokinetic parameters is selected from the group consisting of: a Tₘₐₓ of from about 0.93 hours to about 1.46 hours when the dose of methotrexate is about 20 mg; a half-life of from about 2.86 hours to about 4.47 hours when the dose of methotrexate is about 20 mg; and a combination thereof.

12. The method of claim 2, wherein said set of one or more pharmacokinetic parameters is selected from the group consisting of: a Tₘₐₓ of from about 0.98 hours to about 1.54 hours when the dose of methotrexate is about 25 mg; a half-life of from about 3.02 hours to about 4.72 hours when the dose of methotrexate is about 25 mg.

13. The method of claim 5, wherein said AUC is selected from the group consisting of: AUC₍₀₋ₜ₎ of about 1141 ng*hr/ml; AUC₍₀₋₂₄₎ of about 1150 ng*hr/ml; AUC_{(0-inf)} of about 1161 ng*hr/ml; and combinations thereof.

14. The method of claim 6, wherein said AUC is selected from the group consisting of AUC₍₀₋ₜ₎ of about 1945 ng*hr/ml; AUC₍₀₋₂₄₎ of about 1948 ng*hr/ml; AUC_{(0-inf)} of about 1979 ng*hr/ml; and combinations thereof.

15. The method of claim 7, wherein said AUC is selected from the group consisting of: AUC₍₀₋ₜ₎ of about 2188 ng*hr/ml; AUC₍₀₋₂₄₎ of about 2188 ng*hr/ml; AUC_{(0-inf)} of about 2219 ng*hr/ml; and combinations thereof.

16. The method of claim 8, wherein said AUC is selected from the group consisting of: AUC₍₀₋ₜ₎ of about 2799 ng*hr/ml; an AUC₍₀₋₂₄₎ of about 2799 ng*hr/ml; an AUC_{(0-inf)} of about 2836 ng*hr/ml; and combinations thereof.

17. The method of claim 9, wherein said set of one or more pharmacokinetic parameters is selected from the group consisting of: a Tₘₐₓ of about 1.33 hours; a half-life of about 3 hours.

18. The method of claim 10, wherein said set of one or more pharmacokinetic parameters is selected from the group consisting of: a Tₘₐₓ of about 1.25 hours; a half-life of about 3.68 hours; and combinations thereof.

19. The method of claim 11, wherein said Tₘₐₓ is about 1.17 hours; said half-life is about 3.58 hours.

20. The method of claim 12, wherein said Tₘₐₓ is about 1.23 hours; said half-life is about 3.78 hours.

21. The method of claim 1, wherein said methotrexate is present in an amount ranging from about 5 mg to about 10 mg, from about 5 mg to about 12.5 mg, from about 5 mg to about 15 mg, from about 5 mg to about 17.5 mg, from about 5 mg to about 20 mg, from about 5 mg to about 22.5 mg, from about 5 mg to about 25 mg, from about 5 mg to about 30 mg, from about 5 mg to about 40 mg, 7.5 mg to about 10 mg, from about 7.5 mg to about 12.5 mg, from about 7.5 mg to about 15 mg, from about 7.5 mg to about 17.5 mg, from about 7.5 mg to about 20 mg, from about 7.5 mg to about 22.5 mg, from about 7.5 mg to about 25 mg, from about 7.5 mg to about 30 mg, from about 7.5 mg to about 40 mg, from about 7.5 mg to 50 mg, from about 10 mg to about 12.5 mg, from 10 mg to about 15 mg, from about 10 mg to about 17.5 mg, from about 10 mg to about 20 mg, from about 10 mg to about 22.5 mg, from about 10 mg to about 25 mg, from about 10 to about 30 mg, from about 10 mg to about 40 mg, from about 15 mg to about 17.5 mg, from about 15 mg to about 20 mg, from about 15 mg to about 22.5 mg, from about 15 mg to about 25 mg, from about 15 to about 30 mg, from about 15 to about 35 mg, from about 15 mg to about 40 mg, from about 15 mg to about 35 mg, from about 15 mg to about 50 mg, from about 20 mg to about 25 mg, from about 22.5 mg to about 25 mg, from about 20 to about 30 mg, from about 20 to about 35 mg, from about 20 mg to about 40 mg, from about 20 mg to about 50 mg, from about 22.5 to about 30 mg, from about 22.5 to about 35 mg, from about 22.5 mg to about 40 mg, from about 22.5 mg to about 50 mg, from about 25 to about 30 mg, from about 25 to about 35 mg, from about 25 mg to about 40 mg, from about 25 mg to about 50 mg, from about 25 to about 30 mg, from about 25 to about 35 mg, from about 25 mg to about 40 mg, from about 25 mg to about 50 mg, from about 30 to about 35 mg, from about 30 mg to about 40 mg, from about 30 mg to about 50 mg, or from about 35 mg to about 50 mg.

22. The method of claim 1, wherein said autoimmune disorder is selected from the group consisting of Juvenile idiopathic arthritis (JIA), Juvenile rheumatoid arthritis (JRA), Psoriatic arthritis (PA), and Rheumatoid arthritis (RA).

23. The method of claim 1, wherein said methotrexate is administered in combination with a set of one or more biologics.

24. The method of claim 23, wherein said set of one or more biologics comprises one or more alpha TNFs inhibitors.

25. The method of claim 23, wherein said set of one or more biologics comprises Etanercept (or Enbrel) or infliximab (or Remicade) or a combination thereof.

26. The method of claim 1, wherein said method provides a pharmacokinetic profile that increases linearly in proportion to increases in methotrexate dose level.
